(19) United States
(12) Patent Application Publication
(10) Pub. No.: US 2020/0010439 A1
(43) Pub. Date: Jan. 9, 2020

(54) PROCESSES FOR PREPARING PARTIALLY FLUORINATED EPOXIDES AND PERFLUORINATED EPOXIDES AND COMPOSITIONS RELATED THERETO

(71) Applicant: THE CHEMOURS COMPANY FC, LLC, WILMINGTON, DE (US)

(72) Inventors: VIACHESLAV A PETROV, Hockessin, DE (US); Drew Richard Brandt, West Chester, PA (US)

(73) Assignee: THE CHEMOURS COMPANY FC, LLC, WILMINGTON, DE (US)

(21) Appl. No.: 16/491,234

(22) PCT Filed: Mar. 9, 2018
(86) PCT No.: PCT/US2018/021841

§ 371 Date: Sep. 5, 2019

Related U.S. Application Data
(60) Provisional application No. 62/469,673, filed on Mar. 10, 2017.

(51) Int. Cl. C07D 303/48; C07D 301/03

(52) U.S. Cl. CPC C07D 303/48; C07D 301/03

(57) ABSTRACT

This application relates to the preparation of partially fluorinated epoxides and perfluorinated epoxides which may be useful in various applications including refrigerants, heat transfer media, high-temperature heat pumps, organic Rankine cycles, fire extinguishing/fire suppression, propellants, foam blowing, solvents, gaseous dielectrics, and/or cleaning fluids. Compositions comprising the fluorinated epoxide compounds are also provided.

### CROSS-REFERENCE TO RELATED APPLICATIONS

This application claims the benefit of U.S. Provisional Application Ser. No. 62/469,673, filed Mar. 10, 2017, the disclosure of which is incorporated herein by reference in its entirety.

### TECHNICAL FIELD

This application relates to the preparation of partially fluorinated epoxides, perfluorinated epoxides, and compositions which are useful in applications including refrigerants, heat transfer media, high-temperature heat pumps, organic Rankine cycles, fire extinguishing/fire suppression, propellants, foam blowing, solvents, gaseous dielectrics, and/or cleaning fluids.

### BACKGROUND

Many current commercial propellant, fire suppression and foam blowing applications employ HCFCs or HFCs. HCFCs, due to their Cl content, contribute to ozone depletion and are scheduled for eventual phaseout under the Montreal Protocol. HFCs, while not contributing to ozone depletion, can contribute to global warming, and the use of such compounds has come under scrutiny by environmental regulators. Thus, there is a need for aerosol propellants, fire suppression agents, foam blowing agents, refrigerants, solvents, cleaning and cleaning fluids that are characterized by a zero ozone depletion potential (ODP) and low impact on global warming. This application addresses this need for alternative materials by providing processes for making new compositions in higher yields and purity.

### SUMMARY

The present application describes partially fluorinated and perfluorinated epoxides that are useful for applications including refrigerants, high-temperature heat pumps, organic Rankine cycles, fire extinguishing/fire suppression, propellants, foam blowing, solvents, and/or cleaning fluids. In order to prepare these compounds, the present application provides a process of preparing partially fluorinated and perfluorinated epoxides. The processes described herein utilize an aqueous hypohalite salt (e.g., NaOCl) in the presence of a cationic phase transfer catalyst (e.g., a quaternary ammonium or phosphonium salt) in the presence of an organic solvent (e.g., acetonitrile, toluene or a xylene). Surprisingly, it was found that the combination of a phase transfer catalyst with a solvent improved the conversion of the fluoroalkene to fluoroepoxide from <5% to 95% or greater (see e.g., Examples 18-20). Further, the process described herein is stereospecific and proceeds stereoselectively with formation of either trans- or cis-epoxides of Formula (I), depending on the double bond geometry of starting fluoroolefins. This enables the formation of compounds of Formula (I) in high conversion with conservation of stereochemistry. This represents an advantage over previous processes (see e.g, Kolenko et al, Izv. AN USSR. Ser. Khim. No11, pp. 2509-25-12) that had slow reaction rates and relatively low conversion, enabling the synthesis of less epoxides from less reactive fluoroalkenes, such as internal partially fluorinated olefins.

Accordingly, the present application provides, inter alia, a process of preparing a compound of Formula (I):

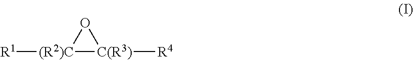

comprising reacting a compound of Formula (II):

R¹—C(R²)═C(R³)—R⁴ (II)

with an aqueous hypohalite salt in the presence of a cationic phase transfer catalyst and an organic solvent, wherein, variables R¹, R², R³, and R⁴ are defined herein.

The present application further provides compounds of Formula (I) and compositions comprising a compound of Formula (I) and a compound of Formula (III):

R⁵—C(R⁶)═C(R⁷)—R⁸ (III)

wherein variables R⁵, R⁶, R⁷, and R⁸ are defined herein, having a high percentage of the compound of Formula (I).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DETAILED DESCRIPTION

The present application describes, inter alia, a process for the preparation for epoxides of fluorinated olefins, which is based on the oxidation of the corresponding olefins with an aqueous hypohalite salt in the presence of an cationic phase transfer catalyst and an organic solvent, as well as compositions and compounds produced therefrom.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Definitions, Abbreviations, and Acronyms

For the terms “for example” and “such as” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “substantially isolated” means that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds are routine in the art. In some embodiments, the compounds provided herein are substantially isolated.

As used herein, the term “alkali metal” refers to sodium, lithium, potassium, or rubidium. In some embodiments, the alkali metal is sodium or potassium.

As used herein, the term “alkali earth metal” refers to beryllium, magnesium, or calcium. In some embodiments, the alkali earth metal is magnesium or calcium.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl” refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tent-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group has 1 to 20, 1 to 18, 1 to 15, 1 to 12, 1 to 10, 1 to 6, 1 to 4, 1 to 3, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 1 to 20, 1 to 18, 1 to 15, 1 to 12, 1 to 10, 1 to 6, 1 to 4, 1 to 3, or 1 to 2 carbon atoms.

As used herein, the term “Cₙ₋ₘ alkoxy” refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tent-butoxy), and the like. In some embodiments, the alkoxy group has 1 to 20, 1 to 18, 1 to 15, 1 to 12, 1 to 10, 1 to 6, 1 to 4, 1 to 3, or 1 to 2 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 20, 1 to 18, 1 to 15, 1 to 12, 1 to 10, 1 to 6, 1 to 4, 1 to 3, or 1 to 2 carbon atoms. Examples of alkylamino groups include, but are not limited to, N-methylamino, N-ethylamino, N-propylamino (e.g., N-(n-propyl)amino and N-i sopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 20, 1 to 18, 1 to 15, 1 to 12, 1 to 10, 1 to 6, 1 to 4, 1 to 3, or 1 to 2 carbon atoms.

As used herein, the term “aryl” refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl group has from 6 to 14 carbon atoms. In some embodiments, the aryl group is phenyl.

As used herein, “halo” refers to F, Cl, Br, or I. In some embodiments, a halo is F, Cl, or Br.

As used herein, “halide” refers to fluoride, chloride, bromide, or iodide. In some embodiments, a halide is chloride or bromide.

As used herein, the term “hypohalite” or “hypohalite salt” refers to a compound of formula M(OX)ₙ, where M is an alkali metal (e.g., sodium or potassium) or an alkali earth metal (e.g., calcium or barium), OX is a hypohalite ion (e.g., OCl⁻ OBr⁻), and n is 1 or 2. Example hypohalite salts include, but are not limited to, sodium hypochlorite, sodium hypobromite, potassium hypochlorite, calcium hypochlorite, and the like.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only (i.e. a partially fluorinated alkoxy or a perfluorinated alkoxy). In some embodiments, the haloalkoxy group has 1 to 20, 1 to 18, 1 to 15, 1 to 12, 1 to 10, 1 to 6, 1 to 4, 1 to 3, or 1 to 2 carbon atoms.

As used herein, the term “Cₙ₋ₘ haloalkyl” refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only (i.e., a partially fluorinated alkyl or a perfluorinated alkyl). In some embodiments, the haloalkyl group has 1 to 20, 1 to 18, 1 to 15, 1 to 12, 1 to 10, 1 to 6, 1 to 4, 1 to 3, or 1 to 2 carbon atoms.

As used herein, the term “partially fluorinated Cₙ₋ₘ alkyl” refers to a linear or branched alkyl group having from one halogen atom to less than 2s+1 halogen atoms which may be the same or different, where “s” is the number of carbon atoms in the alkyl group, and wherein the alkyl group has n to m carbon atoms. Examples of partially fluorinated Cₙ₋ₘ alkyl groups include, but are not limited to, —CH₂F, —CHF₂, —CH₂CH₂F, —CH₂CHF₂, —CH₂CF₃, —CH₂CH₂CF₃, —CH₂CF₂CF₃, —CF₂CF₂CHF₂, and the like. In some embodiments, the partially fluorinated alkyl group has 1 to 20, 1 to 18, 1 to 15, 1 to 12, 1 to 10, 1 to 6, 1 to 4, 1 to 3, or 1 to 2 carbon atoms.

As used herein, the term “perfluorinated Cₙ₋ₘ alkyl” refers to a linear or branched alkyl group having 2s+1 fluorine atoms, where “s” is the number of carbon atoms in the alkyl group, and wherein the alkyl group has n to m carbon atoms. Examples of perfluorinated alkyl groups include, but are not limited to, —CF₃, —CF₂CF₃, —CF₂CF₂CF₃, —CF₂CF₂CF₂CF₃, —C(F)(CF₃)₂, and the like. In some embodiments, the perfluorinated alkyl group has 1 to 20, 1 to 18, 1 to 15, 1 to 12, 1 to 10, 1 to 6, 1 to 4, 1 to 3, or 1 to 2 carbon atoms.

As used herein, the term “partially fluorinated Cₙ₋ₘ alkoxy” refers to a group of formula —O-fluoroalkyl, wherein the fluoroalkyl is a linear or branched partially fluorinated alkyl group having n to m carbon atoms. Examples of partially fluorinated alkoxy groups include, but are not limited to, —OCH₂F, —OCHF₂, —OCH₂CH₂F, —OCH₂CHF₂, —OCH₂CF₃, —OCH₂CH₂CF₃, —OCH₂CF₂CF₃, —OCF₂CF₂CHF₂, and the like. In some embodiments, the partially fluorinated alkoxy group has 1 to 20, 1 to 18, 1 to 15, 1 to 12, 1 to 10, 1 to 6, 1 to 4, 1 to 3, or 1 to 2 carbon atoms.

As used herein, the term “perfluorinated Cₙ₋ₘ alkyl” refers to a group of formula —O-fluoroalkyl, wherein the fluoroalkyl group is a linear or branched perfluoroalkyl group having n to m carbon atoms. Examples of perfluorinated alkyl groups include, but are not limited to, —CF₃, —CF₂CF₃, —CF₂CF₂CF₃, —CF₂CF₂CF₂CF₃, —C(F)(CF₃)₂, and the like. In some embodiments, the perfluorinated alkyl group has 1 to 20, 1 to 18, 1 to 15, 1 to 12, 1 to 10, 1 to 6, 1 to 4, 1 to 3, or 1 to 2 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, 12, 13, or 14 ring-forming carbons (C₃₋₁₄). In some embodiments, the cycloalkyl is a C₃₋₁₄ monocyclic or bicyclic cyclocalkyl. In some embodiments, the cycloalkyl is a C₃₋₇ monocyclic cycloalkyl. In some embodiments, the cycloalkyl is a C₄₋₆ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more ring members selected from C(O), S(O), C(S), S(O)₂, and S(NH)(O). In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more ring members selected from S(O)₂ and S(NH)(O).

As used herein, the term “azeotropic composition” shall be understood to mean a composition where at a given temperature at equilibrium, the boiling point pressure (of the liquid phase) is identical to the dew point pressure (of the vapor phase), i.e., X₂═Y₂. One way to characterize an azeotropic composition is that the vapor produced by partial evaporation or distillation of the liquid has the same composition as the liquid from which it was evaporated or distilled, that is, the admixture distills/refluxes without compositional change. Constant boiling compositions are characterized as azeotropic because they exhibit either a maximum or minimum boiling point, as compared with that of the non-azeotropic mixtures of the same components. Azeotropic compositions are also characterized by a minimum or a maximum in the vapor pressure of the mixture relative to the vapor pressure of the neat components at a constant temperature.

As used herein, the terms “azeotrope-like composition” and “near-azeotropic composition” shall be understood to mean a composition wherein the difference between the bubble point pressure (“BP”) and dew point pressure (“DP”) of the composition at a particular temperature is less than or equal to 5 percent based upon the bubble point pressure, i.e., [(BP−DP)/BP]×100≤5.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100-year time horizon is commonly the value referenced.

As used herein the term “Ozone depletion potential” (ODP) is defined in “The Scientific Assessment of Ozone Depletion, 2002, A report of the World Meteorological Association's Global Ozone Research and Monitoring Project,” section 1.4.4, pages 1.28 to 1.31 (see first paragraph of this section). ODP represents the extent of ozone depletion in the stratosphere expected from a compound on a mass-for-mass basis relative to fluorotrichloromethane (CFC-11).

Refrigeration capacity (i.e., cooling capacity) is a term to define the change in enthalpy of a refrigerant or working fluid in an evaporator per unit mass of refrigerant or working fluid circulated. Volumetric cooling capacity refers to the amount of heat removed by the refrigerant or working fluid in the evaporator per unit volume of refrigerant vapor exiting the evaporator. The refrigeration capacity is a measure of the ability of a refrigerant, working fluid or heat transfer composition to produce cooling. Therefore, the higher the volumetric cooling capacity of the working fluid, the greater the cooling rate that can be produced at the evaporator with the maximum volumetric flow rate achievable with a given compressor. Cooling rate refers to the heat removed by the refrigerant in the evaporator per unit time.

Similarly, volumetric heating capacity is a term to define the amount of heat supplied by the refrigerant or working fluid in the condenser per unit volume of refrigerant or working fluid vapor entering the compressor. The higher the volumetric heating capacity of the refrigerant or working fluid, the greater the heating rate that is produced at the condenser with the maximum volumetric flow rate achievable with a given compressor.

A body to be cooled or heated may be defined as any space, location, object or body for which it is desirable to provide cooling or heating. Examples include spaces (open or enclosed) requiring air conditioning, cooling, or heating, such as a room, an apartment, or building, such as an apartment building, university dormitory, townhouse, or other attached house or single family home, hospitals, office buildings, supermarkets, college or university classrooms or administration buildings and automobile or truck passenger compartments.

Coefficient of performance (COP) is the amount of heat removed in the evaporator divided by the energy required to operate the compressor. The higher the COP, the higher the energy efficiency. COP is directly related to the energy efficiency ratio (EER), that is, the efficiency rating for refrigeration or air conditioning equipment at a specific set of internal and external temperatures.

As used herein, a heat transfer medium comprises a compound or composition used to carry heat from a body to be cooled to a chiller evaporator or from a chiller condenser to a cooling tower or other configuration where heat can be rejected to the ambient.

As used herein, a working fluid comprises a compound or mixture of compounds that function to transfer heat in a cycle wherein the working fluid undergoes a phase change from a liquid to a gas and back to a liquid in a repeating cycle.

Subcooling is the reduction of the temperature of a liquid below that liquid's saturation point for a given pressure. The saturation point is the temperature at which a vapor composition is completely condensed to a liquid (also referred to as the bubble point). But subcooling continues to cool the liquid to a lower temperature liquid at the given pressure. By cooling a liquid below the saturation temperature, the net refrigeration capacity can be increased. Subcooling thereby improves refrigeration capacity and energy efficiency of a system. Subcool amount is the amount of cooling below the saturation temperature (in degrees) or how far below its saturation temperature a liquid composition is cooled.

Superheat is a term that defines how far above the saturation vapor temperature of a vapor composition a vapor composition is heated. Saturation vapor temperature is the temperature at which, if a vapor composition is cooled, the first drop of liquid is formed, also referred to as the “dew point”.

As used herein, the term “incumbent refrigerant” shall be understood to mean the refrigerant for which the heat transfer system was designed to operate, or the refrigerant that is resident in the heat transfer system.

By “in the vicinity of” is meant that the evaporator of the system containing the refrigerant composition is located either within or adjacent to the body to be cooled, such that air moving over the evaporator would move into or around the body to be cooled. In the process for producing heating, “in the vicinity of” means that the condenser of the system containing the refrigerant composition is located either within or adjacent to the body to be heated, such that the air moving over the evaporator would move into or around the body to be heated.

As used herein, the term “Critical Pressure” refers to the pressure at or above which a fluid does not undergo a vapor-liquid phase transition no matter how much the temperature is varied.

The term “extinguishment” is usually used to denote complete elimination of a fire;

whereas, “suppression” is often used to denote reduction, but not necessarily total elimination, of a fire or explosion. As used herein, terms “extinguishment” and “suppression” will be used interchangeably.

Flammability is a term used to mean the ability of a composition to ignite and/or propagate a flame. For refrigerants and other heat transfer compositions, the lower flammability limit (“LFL”) is the minimum concentration of the heat transfer composition in air that is capable of propagating a flame through a homogeneous mixture of the composition and air under test conditions specified in ASTM (American Society of Testing and Materials) E681. The upper flammability limit (“UFL”) is the maximum concentration of the heat transfer composition in air that is capable of propagating a flame through a homogeneous mixture of the composition and air under the same test conditions.

As used herein, the term “lubricant” refers to any material added to a composition or a compressor (and in contact with any heat transfer composition in use within any heat transfer system) that provides lubrication to the compressor to aid in preventing parts from seizing.

As used herein, the term “compatibilizers” refers to compounds which improve solubility of the hydrofluorocarbon of the disclosed compositions in heat transfer system lubricants. In some embodiments, the compatibilizers improve oil return to the compressor. In some embodiments, the composition is used with a system lubricant to reduce oil-rich phase viscosity.

As used herein, “ultra-violet” dye is defined as a UV fluorescent or phosphorescent composition that absorbs light in the ultra-violet or “near” ultra-violet region of the electromagnetic spectrum. The fluorescence produced by the UV fluorescent dye under illumination by a UV light that emits at least some radiation with a wavelength in the range of from 10 nanometers to about 775 nanometers may be detected.

### Chemicals, Abbreviations, and Acronyms

ACN: acetonitrile
ATL: atmospheric life time
CFO-1316mxx: CF₃CCl═CClCF₃
HFO: hydrofluoroolefins
HFO-1336mzz: CF₃CH═CHCF₃
F12E: CF₃CH═CHC₂F₅
F13Ei: CF₃CH═CHCF(CF₃)₂
F33E: C₃F₇CH═CHC₃F₇
F24E: C₂F₅CH═CHC₄F₉
F44E: C₄F₉CH═CHC₄F₉
HFIBO: hexafluoroisobutene oxide, 2,2-bis(trifluoromethyl)oxirane
NaOCl: sodium hypochlorite
PFP-2: perfluoropentene-2
PTC: phase transfer catalysis or phase transfer catalyst

### IUPAC Names for Epoxides

Z-1234ze Epoxide: cis-2-fluoro-3-(trifluoromethyl)oxirane
E-1234ze Epoxide: trans-2-fluoro-3-(trifluoromethyl)oxirane
E-1336mzz Epoxide: trans-2,3-bis(trifluoromethyl)oxirane
Z-1336mzz Epoxide: cis-2,3-bis(trifluoromethyl)oxirane
F12E Epoxide: trans-2,3-bis(trifluoromethyl)oxirane
F-13Ei Epoxide: trans-2-(trifluoromethyl)-3-(perfluoropropan-2-yl)oxirane
F-33E Epoxide: trans-2,3-bis(perfluoropropyl)oxirane
F-24E Epoxide: trans-2-(perfluorobutyl)-3-(perfluoroethyl)oxirane
F-44E Epoxide: trans-2,3-bis(perfluorobutyl)oxirane
HFX-90 Epoxide: 2-(2,2,2-trifluoroethoxy)-3-fluoro-2-(trifluoromethyl)-3-(perfluoroethyl)oxirane
1316mxx Epoxide: 2,3-dichloro-2,3-bis(trifluoromethyl)oxirane
E-1438ezy Epoxide: trans-2-fluoro-3-(perfluoropropan-2-yl)oxirane
Z-1438ezy Epoxide: cis-2-fluoro-3-(perfluoropropan-2-yl)oxirane
1,2-H-Hexafluorocyclopentene Epoxide: 2,2,3,3,4,4-hexafluoro-6-oxa-bicyclo[3 1.0]hexane
Perfluoroheptene-3 Epoxide: 2,3-difluoro-2-(perfluoroethyl)-3-(perfluoropropyl)oxirane
Perfluorooctene-2 Epoxide: 2,3-difluoro-2-(trifluoromethyl)-3-(perfluoropentyl)oxirane

### IUPAC Names for Olefins

Z-1234ze: (Z)-1,3,3,3-tetrafluoroprop-1-ene
E-1234ze: (E)-1,3,3,3-tetrafluoroprop-1-ene
E-1336mzz: (E)-1,1,1,4,4,4-hexafluorobut-2-ene
Z-1336mzz: (Z)-1,1,1,4,4,4-hexafluorobut-2-ene
F12E: (E)-1,1,1,4,4,5,5,5-octafluoropent-2-ene
F-13Ei: (E)-1,1,1,5,5,5-hexafluoro-4,4-bis(trifluoromethyl)pent-2-ene
F-33E: (E)-1,1,1,2,2,3,3,6,6,7,7,8,8,8-tetradecafluorooct-4-ene
F-24E: (E)-1,1,1,2,2,5,5,6,6,7,7,8,8,8-tetradecafluorooct-3-ene
F-44E: (E)-1,1,2,2,3,3,4,4,7,7,8,8,9,9,10,10,10-octadecafluorodec-5-ene
HFX-90: 2-(2,2,2-trifluoroethoxy)-1,1,1,4,4,5,5,5-octafluoropent-2-ene
1316mxx: (E/Z)-2,3-dichloro-1,1,1,4,4,4-hexafluorobut-2-ene
E-1438ezy: (E)-1,3,4,4,4-pentafluoro-3-(trifluoromethyl)but-1-ene
Z-1438ezy: (Z)-1,3,4,4,4-pentafluoro-3-(trifluoromethyl)but-1-ene
1,2-H-Hexafluorocyclopentene: 3,3,4,4,5,5-hexafluorocyclopent-1-ene
Perfluoropenetene-2: perfluoropent-2-ene
Perfluoroheptene-3: perfluorohept-3-ene
Perfluorooctene-2: perfluorooct-2-ene

### Processes of Preparing Partially Fluorinated and Perfluorinated Epoxides

The present application provides a process of preparing a compound of Formula (I):

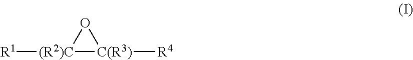

comprising reacting a compound of Formula (II):

R¹—C(R²)═C(R³)—R⁴ (II)

with an aqueous hypohalite salt in the presence of a cationic phase transfer catalyst and an organic solvent, wherein:

R¹ and R⁴ are each independently H, Cl, F, Br, I, a partially fluorinated C₁₋₄ alkoxy, or a perfluorinated C₁₋₄ alkoxy;

R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;

wherein at least one of R¹, R², and R⁴ is not H; and

R³ is selected from partially fluorinated and perfluorinated C₁₋₁₀ alkyl; or alternatively, R² and R³ are each independently selected from partially fluorinated or perfluorinated C₁₋₅ akylene, which together form a monocyclic ring.

In some embodiments, the present application provides a process of preparing a compound of Formula (I):

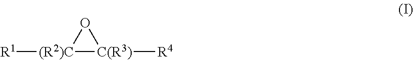

comprising reacting a compound of Formula (II):

R¹—C(R²)═C(R³)—R⁴ (II)

with an aqueous hypohalite salt in the presence of a cationic phase transfer catalyst and an organic solvent, wherein:

R¹ and R⁴ are each independently H, Cl, F, a partially fluorinated C₁₋₄ alkoxy, or a perfluorinated C₁₋₄ alkoxy; and

R² and R³ are each independently selected from partially fluorinated or perfluorinated C₁₋₁₀ alkyl;

or alternatively, R² and R³ are each independently selected from partially fluorinated or perfluorinated C₁₋₅ alkylene, which together form a monocyclic ring.

In some embodiments, the compound of Formula (I) is the cis-isomer. In some embodiments, the compound of Formula (I) is the trans-isomer. In some embodiments, the compound of Formula (II) is the cis-isomer. In some embodiments, the compound of Formula (II) is the trans-isomer.

In some embodiments, the compounds are non-cyclic. Accordingly, in some embodiments, R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₁₀ alkyl.

In some embodiments of the non-cyclic compounds, R¹ and R⁴ are identical. In some embodiments of the non-cyclic compounds, R¹ and R⁴ are different. In some embodiments of the non-cyclic compounds, R¹ and R⁴ are each H. In some embodiments of the non-cyclic compounds, R¹ and R⁴ are each F. In some embodiments of the non-cyclic compounds, R¹ and R⁴ are each Cl. In some embodiments of the non-cyclic compounds, R¹ is a partially fluorinated C₁₋₄ alkoxy and R⁴ is H.

In some embodiments of the non-cyclic compounds, R² and R³ are identical. In some embodiments of the non-cyclic compounds, R² and R³ are different. In some embodiments of the non-cyclic compounds, R² and R³ are each independently selected from partially fluorinated or perfluorinated C₁₋₁₀ alkyl. In some embodiments of the non-cyclic compounds, R² and R³ are each an independently selected perfluorinated C₁₋₁₀ alkyl. In some embodiments of the non-cyclic compounds, R² and R³ are each an independently selected partially fluorinated C₁₋₁₀ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated C₁₋₆ alkyl. In some embodiments of the non-cyclic compounds, R² and R³ are each independently selected from perfluorinated C₁₋₆ alkyl. In some embodiments of the non-cyclic compounds, R² and R³ are each independently CF₃, CF₂CF₃, CF(CF₃)₂, CF₂CF₂CF₃, CF₂CF₂CF₂CF₃, or CF₂CF₂CF₂CF₂CF₃.

In some embodiments:
R¹ and R⁴ are H;
R² is partially fluorinated or perfluorinated C₁₋₁₀ alkyl; and
R³ is partially fluorinated or perfluorinated C₁₋₁₀ alkyl.

In some embodiments:
R¹ and R⁴ are H;
R² is partially fluorinated or perfluorinated C₁₋₆ alkyl; and
R³ is partially fluorinated or perfluorinated C₁₋₆ alkyl.

In some embodiments:
R¹ and R⁴ are H;
R²is selected from CF₃, CF₂CF₃, CF(CF₃)₂, CF₂CF₂CF₃, CF₂CF₂CF₂CF₃, or CF₂CF₂CF₂CF₂CF₃;
R³ is selected from CF₃, CF₂CF₃, CF(CF₃)₂, CF₂CF₂CF₃, CF₂CF₂CF₂CF₃, or CF₂CF₂CF₂CF₂CF₃.

In some embodiments, the compounds are cyclic. Accordingly, in some embodiments, R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₅ alkylene, which together form a monocyclic ring.

In some embodiments of the cyclic compounds, R¹ and R⁴ are identical. In some embodiments of the cyclic compounds, R¹ and R⁴ are different. In some embodiments of the cyclic compounds, R¹ and R⁴ are each H. In some embodiments of the cyclic compounds, R¹ and R⁴ are each F. In some embodiments of the cyclic compounds, R¹ and R⁴ are each Cl. In some embodiments of the cyclic compounds, R¹ is a partially fluorinated C₁₋₄ alkoxy and R⁴ is H.

In some embodiments, R² and R³ are each independently selected from partially fluorinated or perfluorinated C₁₋₅ alkylene, which together form a monocyclic ring. In some embodiments, R² and R³ are each independently selected from a perfluorinated C₁₋₅ alkylene, which together form a monocyclic ring. In some embodiments, R² and R³, together with the carbon atoms to which they are attached, form a 4-6 membered monocyclic ring. In some embodiments, R¹ and R⁴ are each independently H or F; and R² and R³ are each independently selected from partially fluorinated or perfluorinated C₁₋₅ alkylene, which together form a monocyclic ring. In some embodiments, R¹ and R⁴ are each independently H or F; and R² and R³ are each an independently selected perfluorinated C₁₋₅ alkylene, which together form a monocyclic ring. In some embodiments, R¹ and R⁴ are each independently H or F; and R² and R³ are each independently selected from partially fluorinated or perfluorinated C₁₋₁₀ alkyl.

In some embodiments:
R¹ and R⁴ are H;
R² is partially fluorinated or perfluorinated C₁₋₅ alkyl; and
R³ is partially fluorinated or perfluorinated C₁₋₅ alkyl;
wherein said R² and R³ are taken together form a monocyclic ring and R⁶ and R⁷ are taken together form a monocyclic ring.

In some embodiments:
R¹ and R⁴ are H;
R² is partially fluorinated or perfluorinated C₂₋₃ alkyl; and
R³ is partially fluorinated or perfluorinated C₂₋₃ alkyl;
wherein said R² and R³ are taken together form a monocyclic ring and R⁶ and R⁷ are taken together form a monocyclic ring.

In some embodiments:
R¹ and R⁴ are H;
R² is selected from —CF₂CF₂— and —CF₂CF₂CF₂—;
R³ and R⁷ are identical and are selected from —CF₂CF₂— and —CF₂CF₂CF₂—;
wherein said R² and R³ are taken together form a monocyclic ring and R⁶ and R⁷ are taken together form a monocyclic ring;

In some embodiments, the compound of Formula (I) is selected from the group consisting of:
2,3-difluoro-2-(trifluoromethyl)oxirane;
2-fluoro-3-(trifluoromethyl)oxirane;
2,3-bis(trifluoromethyl)oxirane;
2-(trifluoromethyl)-3-(perfluoroethyl)oxirane;
2-(trifluoromethyl)-3-(perfluoropropan-2-yl)oxirane;
2,3-bis(perfluoropropyl)oxirane;
2-(perfluorobutyl)-3-(perfluoroethyl)oxirane;
2,3-bis(perfluorobutyl)oxirane;
2-(2,2,2-trifluoroethoxy)-3-fluoro-2-(trifluoromethyl)-3-(perfluoroethyl)oxirane;
2,3-dichloro-2,3-bis(trifluoromethyl)oxirane;
2-fluoro-3-(perfluoropropan-2-yl)oxirane;
2,2,3,3,4,4-hexafluoro-6-oxa-bicyclo[3.1.0]hexane;
2,2,3,3-tetrafluoro-5-oxabicyclo[2.1.0]pentane;
2,3-difluoro-2-(perfluoroethyl)-3-(perfluoropropyl)oxirane; and
2,3-difluoro-2-(trifluoromethyl)-3-(perfluoropentyl)oxirane.

In some embodiments, the compound of Formula (I) is selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-trifluoroethoxy)-3-fluoro-2-(trifluoromethyl)-3-(perfluoroethyl)oxirane;
(E)-2-(2,2,2-trifluoroethoxy)-3-fluoro-2-(trifluoromethyl)-3-(perfluoroethyl)oxirane;
cis-2,3-dichloro-2,3-bis(trifluoromethyl)oxirane;
trans-2, 3-dichloro-2, 3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-Hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

The compounds of Formula (I) provided herein include stereoisomers of the compounds. All stereoisomers, such as enantiomers and diastereomers, are intended unless otherwise indicated.

The processes provided herein are stereospecific processes. For example, reacting an olefin of Formula (II) having the (E)- or trans-configuration according to a process provided herein will substantially yield a compound of Formula (I) having the (E)- or trans-configuration. Likewise, reacting an olefin of Formula (II) having the (Z)- or cis-configuration according to a process provided herein will substantially yield a compound of Formula (I) having the (Z)- or cis-configuration. In addition, reacting an isomeric mixture of olefins of Formula (II) having the (Z) or cis- and (E) or trans- configurations according to a process provided herein will substantially yield a mixture of compounds of Formula (I) with (Z) or cis- and (E)- or trans-ratios similar to those observed in the corresponding starting material.

In some embodiments, the compound of Formula (I) is a compound of Formula (Ia), (Ib), (Ic), or (Id):

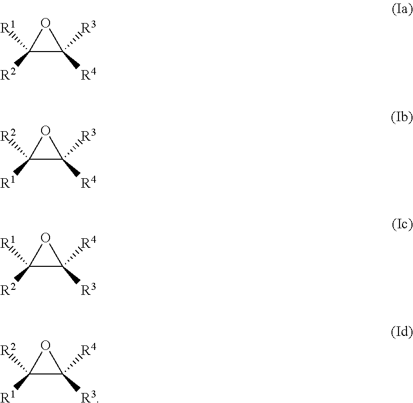

When the compound of Formula (I) described as the trans-isomer, it can be a mixture of compounds of Formula (Ia) and (Id). When the compound of Formula (I) described as the cis-isomer, it can be a mixture of compounds of Formula (Ib) and (Ic).

In some embodiments, the hypohalite salt is an alkali metal hypohalite salt or an alkali earth metal hypohalite salt. In some embodiments, the hypohalite salt is an alkali metal hypohalite salt. In some embodiments, the hypohalite salt is a hypochlorite salt. In some embodiments, the hypohalite salt is selected from NaOCl, KOCl, NaOBr, and Ca(OCl)₂. In some embodiments, the hypohalite salt is KOCl or NaOCl. In some emmbodiments, the hypohalite salt is NaOCl.

In some embodiments, the cationic phase transfer catalyst is a quaternary ammonium salt. Examples of quaternary ammonium salts useful as phase transfer catalysts include, but are not limited to, tricaprylylmethylammonium chloride (Aliquat® 336), tetraethylammonium chloride, tetraethylammonium bromide, tetramethyl ammonium hydroxide, tetrabutylammonium chloride, tetrabutylammonium bromide, tetrabutyl ammonium hydroxide, trimethylbenzylammonium chloride, trimethylbenzylammonium bromide, and trimethylbenzylammonium hydroxide.

In some embodiments, the cationic phase transfer catalyst is a quaternary phosphonium salt. Examples of quaternary phosphonium salts useful as phase transfer catalysts include, but are not limited to, tetra-n-butylphosphonium hydrogensulfate, tetraphenylphosphonium bromide, trans-2-butene-1,4-bis(triphenylphosphonium chloride), (4-bromobenzyl)triphenylphosphonium bromide, tributyl(cyanomethyl)phosphonium chloride, tributyldodecylphosphonium bromide, tributylhexadecylphosphonium bromide, tributyl-n-octylphosphonium bromide, tetrakis(hydroxymethyl)phosphonium chloride, tetrakis(hydroxymethyl)phosphonium sulfate, tetrabutylphosphonium bromide, tetraphenylphosphonium chloride, tetraethylphosphonium bromide, tetrabutylphosphonium chloride, tetra-n-octylphosphonium bromide, tetrabutylphosphonium tetrafluoroborate, and tetrabutylphosphonium hexafluorophosphate

In some embodiments, the cationic phase transfer catalyst has formula (R^{a})(R^{b})(R^{c})(R^{d})N⁺X⁻ or (R^{a})(R^{b})(R^{c})(R^{d})P⁺X⁻, wherein R^{a}, R^{b}, R^{c}, and R^{d} are each independently selected from C₁₋₁₈ alkyl, C₃₋₁₄ cycloalkyl, 4-14 membered heterocycloalkyl, C₆₋₁₄ aryl, 5-14 membered heteroaryl, C₃₋₁₄ cycloalkyl-C₁₋₃ alkylene, 4-14 membered heterocycloalkyl-C₁₋₃ alkylene, C₆₋₁₄ membered aryl-C₁₋₃ alkylene, and 5-14 membered heteroaryl-C₁₋₃ alkylene, each of which is optionally substituted by 1, 2, 3, or 4 groups independently selected from OH, Ci-6 alkoxy, Ci-6 alkyl, C2-6 alkenyl, Ci-6 fluoroalkyl, di-(C₁₋₆ alkyl)amino, C3-14 cycloalkyl, 4-14 membered heterocycloalkyl, C6-14 aryl, and 5-14 membered heteroaryl; and X″ is an anion.

In some embodiments, the cationic phase transfer catalyst has formula (R^{a})(R^{b})(R^{c})(R^{d})N⁺X⁻, wherein R^{a}, R^{b}, R^{c}, and R^{d} are each independently selectedx from C₁₋₁₂ alkyl; and X is a halide ion or HSO₄⁻. In some embodiments, the cationic phase transfer catalyst has formula (R^{a})(R^{b})(R^{c})(R^{d})N⁺X⁻, wherein R^{a}, R^{b}, R^{c}, and R^{d} are each independently selected from C₁₋₁₂ alkyl; and Xis chloride, bromide, or HSO₄⁻.

In some embodiments, the cationic phase transfer catalyst has formula (R^{a})(R^{b})(R^{c})(R^{d})P⁺X⁻, wherein R^{a}, R^{b}, R^{c}, and R^{d} are each independently selected from C₁₋₁₂ alkyl or phenyl; and X is a halide ion or HSO₄⁻. In some embodiments, the cationic phase transfer catalyst has formula (R^{a})(R^{b})(R^{c})(R^{d})P⁺X⁻, wherein R^{a}, R^{b}, R^{c}, and R^{d} are each independently selected from C₁₋₁₂ alkyl or phenyl; and X is chloride, bromide, or HSO₄⁻.

In some embodiments, at least two of R^{a}, R^{b}, R^{c}, and R^{d} are identical. In some embodiments, at least three of R^{a}R^{b}, R^{c}, and R^{d} are identical. In some embodiments, R^{a}, R^{b}, R^{c}, and R^{d} are identical. In some embodiments, each R^{a}, R^{b}, R^{c}, and R^{d} is an independently selected C₁₋₁₀ alkyl. In some embodiments, each R^{a}, R^{b}, R^{c}, and R^{d} is phenyl.

In some embodiments, the cationic phase transfer catalyst is (n-butyl)₄P⁺Br⁻, (n-butyl)₄P⁺HSO₄⁻, (n-butyl)₄N⁺Br⁻, (n-butyl)₄N⁺HSO₄⁻, Ph₄P⁺Br⁻, (ethyl)₄N⁺Br⁻, or Aliquat® 336.

In some embodiments, the solvent is acetonitrile, tetrahydrofuran, diethyl ether, methyl-t-butyl ether, dimethyoxyethane, bis(2-methoxyethyl) ether, or benzene substituted with 1, 2, 3, or 4 independently selected C₁₋₄ alkyl groups. In some embodiments, the solvent is acetonitrile or benzene substituted with 1, 2, 3, or 4 independently selected C₁₋₄ alkyl groups. In some embodiments, the solvent is acetonitrile or benzene substituted with 1 or 2 independently selected C₁₋₄ alkyl groups. In some embodiments, the solvent is acetonitrile or benzene substituted with 1 or 2 independently selected methyl groups. In some embodiments, the solvent is acetonitrile, toluene, o-xylene, m-xylene, p-xylene, or a mixture thereof. In some embodiments, the solvent is acetonitrile. In some embodiments, the solvent is toluene. In some embodiments, the solvent is o-xylene, m-xylene, p-xylene, or a mixture thereof.

In some embodiments, the reaction is conducted at a basic pH, for example, a pH greater than 7 to about 14, greater than 8 to about 14, greater than 9 to about 14, greater than 10 to about 14, greater than 11 to about 14, greater than 12 to about 14, or greater than 13 to about 14. In some embodiments the reaction is conducted at a pH of from about 8 to about 14, about 8 to about 12, about 8 to about 10, about 10 to about 14, about 10 to about 12, or about 12 to about 14. In some embodiments, the reaction is conducted at a pH of from about 10 to about 14. In some embodiments, reaction is conducted at a pH of from about 11 to about 12.

In some embodiments, the reaction is conducted at a temperature of from about 0° C. to about 40° C., for example, from about 0° C. to about 40° C., from about 10° C. to about 40° C., from about 20° C. to about 40° C., from about 30° C. to about 40° C., from about 0° C. to about 30° C., from about 10° C. to about 30° C., from about 20° C. to about 30° C., from about 0° C. to about 20° C., from about 10° C. to about 20° C., or from about 0° C. to about 10° C. In some embodiments, the reaction is conducted at a temperature of from about 10° C. to about 30° C., from about 10° C. to about 20° C., from about 20° C. to about 30° C., or from about 25° C. to about 30° C. In some embodiments, the reaction is conducted at a temperature of from about 10° C. to about 30° C. In some embodiments, the reaction is conducted at a temperature of from about 10° C. to about 20° C. In some embodiments, the reaction is conducted at a temperature of from about 20° C. to about 30° C. In some embodiments, the reaction is conducted at a temperature of from about 25° C. to about 30° C.

In some embodiments, a molar excess of hypohalite salt is used based on one equivalent of the compound of Formula (II), for example, greater than about 2 molar equivalents, greater than about 5 molar equivalents, greater than about 10 molar equivalents, greater than about 20 molar equivalents, greater than about 40 molar equivalents, greater than about 60 molar equivalents, and the like. In some embodiments, about 10 to about 40 molar equivalents of the hypohalite salt is used based on one equivalent of the compound of Formula (II). In some embodiments, about 10 to about 30 molar equivalents of the hypohalite salt is used based on one equivalent of the compound of Formula (II). In some embodiments, about 10 to about 20 molar equivalents of the hypohalite salt is used based on one equivalent of the compound of Formula (II). In some embodiments, about 5 to about 10 molar equivalents of the hypohalite salt is used based on one equivalent of the compound of Formula (II). In some embodiments, about 2 to about 5 molar equivalents of the hypohalite salt is used based on one equivalent of the compound of Formula (II).

In some embodiments, about 1 mol % to about 15 mol % of the cationic phase transfer catalyst is used based on one equivalent of the compound of Formula (II). In some embodiments, about 3 mol % to about 15 mol % of the cationic phase transfer catalyst is used based on one equivalent of the compound of Formula (II). In some embodiments, about 3 mol % to about 10 mol % of the cationic phase transfer catalyst is used based on one equivalent of the compound of Formula (II). In some embodiments, about 5 mol % to about 10 mol % of the cationic phase transfer catalyst is used based on one equivalent of the compound of Formula (II).

In some embodiments, the compound of Formula (II) is a compound of Formula (IIa), (IIb), (IIc), or (IId):

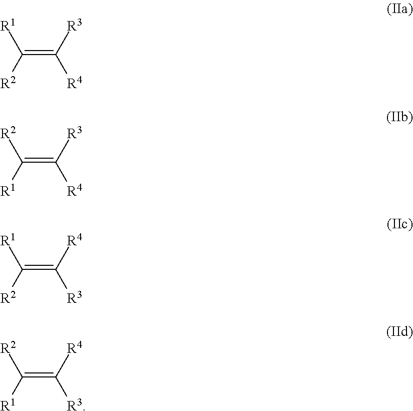

The processes provided herein can be monitored according to any suitable method known in the art. For example, product formation can be monitored by spectroscopic means, such as nuclear magnetic resonance spectroscopy (e.g., ¹H, ¹⁹F or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC), normal phase silica chromatography, distillation, and any combination thereof.

### Compositions

The stereospecific and high conversion processes provided herein allow the formation of compositions having a high amount of the fluoroepoxide as compared to the starting fluoroolefins. This is particularly surprising with respect to internal fluoroolefins that are not fully fluorinated on the olefin carbons and which are not tri-substituted or higher substituted at the olefin positions by perfluoroalkyl groups. Further, when starting from the cis or trans fluoroolefin, the present process provides the corresponding fluoroepoxide with retention of stereochemistry. This preservation of stereochemistry was surprising and unexpected.

Accordingly, in one aspect, the present application provides a composition comprising a compound of Formula I:

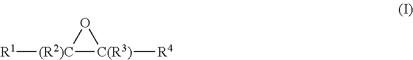

R¹ and R⁴ are each independently H, Cl, F, Br, I, a partially fluorinated C₁₋₄ alkoxy, or a perfluorinated C₁₋₄ alkoxy;
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;

wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring;

wherein the compound of Formula I has the (Z) or (E) configuration and the composition is substantially free of the opposite stereoisomers.

Compounds labeled as trans herein have the (E) configuration, while compounds labeled as cis herein have the (Z) configuration.

For example, for a composition comprising a compound of Formula I having the (Z) configuration, the opposite stereoisomers would be the stereoisomers having the (E) configuration. In some embodiments, substantially free means less than 1% of the opposite stereoisomers. In some embodiments, substantially free means less than 0.5, 0.4, 0.3, 0.2 or 0.1% of the opposite stereoisomers.

Accordingly, the present application further provides a composition comprising a compound of Formula (I):

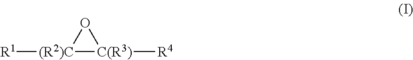

and a compound of Formula (III):

R⁵—C(R⁶)═C(R⁷)—R⁸ (III)

wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are Cl, F, Br, I, partially fluorinated C₁₋₁₀ alkyl, or perfluorinated C₁₋₁₀ alkyl; and
R³ and R⁷ are identical and are partially fluorinated or perfluorinated C₁₋₁₀ alkyl; provided that either R¹ and R⁵ are H or Cl; or R⁴ and R⁸ are H or Cl.

In some embodiments:
the compounds of Formula (I) and Formula (III) have the same stereochemistry;
R¹ and R⁵ are identical and are H, Cl, F, a partially fluorinated C₁₋₄ alkoxy, or a perfluorinated C₁₋₄ alkoxy;
R⁴ and R⁸ are identical and each independently H, Cl, F, a partially fluorinated C₁₋₄ alkoxy, or a perfluorinated C₁₋₄ alkoxy;
R² and R⁶ are identical and are partially fluorinated or perfluorinated C₁₋₁₀ alkyl; and
R³ and R⁷ are identical and are partially fluorinated or perfluorinated C₁₋₁₀ alkyl; provided that either R¹ and R⁵ are H; or R⁴ and R⁸ are H.

In some embodiments, the present application further provides a composition comprising a compound of Formula (I):

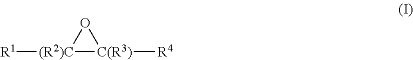

and a compound of Formula (III):

R⁵—C(R⁶)═C(R⁷)—R⁸ (III)

wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are partially fluorinated or perfluorinated C₁₋₅ alkylene; and
R³ and R⁷ are identical and are partially fluorinated or perfluorinated C₁₋₅ alkylene;
wherein said R² and R³ are taken together form a monocyclic ring and R⁶ and R⁷ are taken together form a monocyclic ring.

In some embodiments:
the compounds of Formula (I) and Formula (III) have the same stereochemistry;
R¹ and R⁵ are identical and are H, Cl, F, a partially fluorinated C₁₋₄ alkoxy, or a perfluorinated C₁₋₄ alkoxy;

R⁴ and R⁸ are identical and each independently H, Cl, F, a partially fluorinated C₁₋₄ alkoxy, or a perfluorinated C₁₋₄ alkoxy;
R² and R⁶ are identical and are partially fluorinated or perfluorinated C₁₋₁₀ alkyl;
R³ and R⁷ are identical and are partially fluorinated or perfluorinated C₁₋₁₀ alkyl;
or alternatively, R² and R⁶ are identical and are partially fluorinated or perfluorinated C₁₋₅ alkylene; and R³ and R⁷ are identical and are partially fluorinated or perfluorinated C₁₋₅ alkylene; wherein said R² and R³ are taken together form a monocyclic ring and R⁶ and R⁷ are taken together form a monocyclic ring.

In some embodiments, the composition is a crude reaction mixture before purification.

In some embodiments, the compositions have a molar ratio of the compound of Formula (I) to the compound of Formula (III) of from about 50:50 to about 99.9:0.01, 60:40 to about 99.9:0.01, 70:30 to about 99.9:0.01, 80:20 to about 99.9:0.01, 90:10 to about 99.9:0.01, or 95:05 to about 99.9:0.01. In some embodiments, the molar ratio of the compound of Formula (I) to the compound of Formula (III) in the composition is from about 50:50 to about 99.9:0.01. In some embodiments, the molar ratio of the compound of Formula (I) to the compound of Formula (III) in the composition is from about 80:20 to about 99.9:0.01. In some embodiments, the molar ratio of the compound of Formula (I) to the compound of Formula (III) in the composition is from about 90:10 to about 99.9:0.01.

In some embodiments, at least one of R¹ and R⁵ or R⁴ and R⁸ is not F. In some embodiments, R¹, R⁵, R⁴, and R⁸ are H.

In some embodiments, the composition comprises a compound of Formula (I):

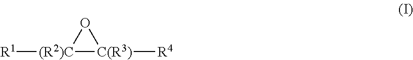

and a compound of Formula (III):

R⁵—C(R⁶)═C(R⁷)—R⁸ (III)

having a molar ratio of the compound of Formula (I) to the compound of Formula (III) of from about 50:50 to about 99.9:0.01;
wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are Cl, F, Br, I, partially fluorinated C₁₋₁₀ alkyl, or perfluorinated C₁₋₁₀ alkyl; and
R³ and R⁷ are identical and are partially fluorinated or perfluorinated C₁₋₁₀ alkyl;
provided that either R¹ and R⁵ are H or Cl; or R⁴ and R⁸ are H or Cl.

In some embodiments, the composition comprises a compound of Formula (I):

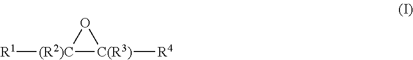

and a compound of Formula (III):

R⁵—C(R⁶)═C(R⁷)—R⁸ (III)

having a molar ratio of the compound of Formula (I) to the compound of Formula (III) of from about 50:50 to about 99.9:0.01; wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are partially fluorinated or perfluorinated C₁₋₁₀ alkyl; and
R³ and R⁷ are identical and are partially fluorinated or perfluorinated C₁₋₁₀ alkyl;
provided that either R¹ and R⁵ are H; or R⁴ and R⁸ are H.

In some embodiments, the compositions have a molar ratio of the compound of Formula (I) to the compound of Formula (III) of from about 50:50 to about 99.9:0.01, 60:40 to about 99.9:0.01, 70:30 to about 99.9:0.01, 80:20 to about 99.9:0.01, 90:10 to about 99.9:0.01, or 95:05 to about 99.9:0.01. In some embodiments, at least one of R¹ and R⁵ or R⁴ and R⁸ is not F. In some embodiments, R¹, R⁵, R⁴, and R⁸ are H.

In some embodiments:
R¹ and R⁵ are H;
R⁴ and R⁸ are H;
R² and R⁶ are identical and are partially fluorinated or perfluorinated C₁₋₁₀ alkyl; and
R³ and R⁷ are identical and are partially fluorinated or perfluorinated C₁₋₁₀ alkyl.

In some embodiments:
R¹ and R⁵ are H;
R⁴ and R⁸ are H;
R² and R⁶ are identical and are partially fluorinated or perfluorinated C₁₋₆ alkyl; and
R³ and R⁷ are identical and are partially fluorinated or perfluorinated C₁₋₆ alkyl.

In some embodiments:
R¹ and R⁵ are H;
R⁴ and R⁸ are H;
R² and R⁶ are identical and are selected from CF₃, CF₂CF₃, CF(CF₃)₂, CF₂CF₂CF₃, CF₂CF₂CF₂CF₃, or CF₂CF₂CF₂CF₂CF₃;
R³ and R⁷ are identical and are selected from CF₃, CF₂CF₃, CF(CF₃)₂, CF₂CF₂CF₃, CF₂CF₂CF₂CF₃, or CF₂CF₂CF₂CF₂CF₃.

In each of preceding embodiments, the compositions have a molar ratio of the compound of Formula (I) to the compound of Formula (III) of from about 80:20 to about 99.9:0.01, 90:10 to about 99.9:0.01, or 95:05 to about 99.9:0.01.

In some embodiments, the composition comprises a compound of Formula (I):

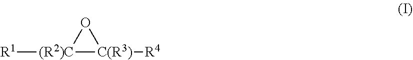

and a compound of Formula (III):

R⁵—C(R⁶)═C(R⁷)—R⁸ (III)

having a molar ratio of the compound of Formula (I) to the compound of Formula (III) of from about 50:50 to about 99.9:0.01;
wherein:

the compounds of Formula (I) and Formula (III) have the same stereochemistry;

R¹ and R⁵ are identical and are H, Cl, F, Br, I, a partially fluorinated C₁₋₄ alkoxy, or a perfluorinated C₁₋₄ alkoxy;

R⁴ and R⁸ are identical and each independently H, Cl, F, Br, I, a partially fluorinated C₁₋₄ alkoxy, or a perfluorinated C₁₋₄ alkoxy;

R² and R⁶ are identical and are partially fluorinated or perfluorinated C₁₋₅ alkylene; and

R³ and R⁷ are identical and are partially fluorinated or perfluorinated C₁₋₅ alkylene; wherein said R² and R³ are taken together form a monocyclic ring and R⁶ and R⁷ are taken together form a monocyclic ring.

In some embodiments, the composition comprises a compound of Formula (I):

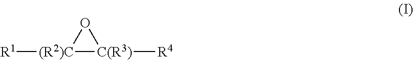

and a compound of Formula (III):

R⁵—C(R⁶)═C(R⁷)—R⁸ (III)

having a molar ratio of the compound of Formula (I) to the compound of Formula (III) of from about 50:50 to about 99.9:0.01;
wherein:
the compounds of Formula (I) and Formula (III) have the same stereochemistry;
R¹ and R⁵ are identical and are H, Cl, F, a partially fluorinated C₁₋₄ alkoxy, or a perfluorinated C₁₋₄ alkoxy;
R⁴ and R⁸ are identical and each independently H, Cl, F, a partially fluorinated C₁₋₄ alkoxy, or a perfluorinated C₁₋₄ alkoxy;
R² and R⁶ are identical and are partially fluorinated or perfluorinated C₁₋₅ alkylene; and
R³ and R⁷ are identical and are partially fluorinated or perfluorinated C₁₋₅ alkylene;
wherein said R² and R³ are taken together form a monocyclic ring and R⁶ and R⁷ are taken together form a monocyclic ring.

In some embodiments, the compositions have a molar ratio of the compound of Formula (I) to the compound of Formula (III) of from about 50:50 to about 99.9:0.01, 60:40 to about 99.9:0.01, 70:30 to about 99.9:0.01, 80:20 to about 99.9:0.01, 90:10 to about 99.9:0.01, or 95:05 to about 99.9:0.01. In some embodiments, at least one of R¹ and R⁵ or R⁴ and R⁸ is not F. In some embodiments, R¹, R⁵, R⁴, and R⁸ are H.

In some embodiments:
R¹ and R⁵ are H;
R⁴ and R⁸ are H;
R² and R⁶ are identical and are partially fluorinated or perfluorinated C₁₋₅ alkyl; and
R³ and R⁷ are identical and are partially fluorinated or perfluorinated C₁₋₅ alkyl;
   wherein said R² and R³ are taken together form a monocyclic ring and R⁶ and R⁷ are taken together form a monocyclic ring.

In some embodiments:
R¹ and R⁵ are H;
R⁴ and R⁸ are H;
R² and R⁶ are identical and are partially fluorinated or perfluorinated C₂₋₃ alkyl; and
R³ and R⁷ are identical and are partially fluorinated or perfluorinated C₂₋₃ alkyl;
   wherein said R² and R³ are taken together form a monocyclic ring and R⁶ and R⁷ are taken together form a monocyclic ring.

In some embodiments:
R¹ and R⁵ are H;
R⁴ and R⁸ are H;
R² and R⁶ are identical and are selected from —CF₂CF₂— and —CF₂CF₂CF₂—;
R³ and R⁷ are identical and are selected from —CF₂CF₂— and —CF₂CF₂CF₂—;
   wherein said R² and R³ are taken together form a monocyclic ring and R⁶ and R⁷ are taken together form a monocyclic ring;

In each of preceding embodiments, the compositions have a molar ratio of the compound of Formula (I) to the compound of Formula (III) of from about 80:20 to about 99.9:0.01, 90:10 to about 99.9:0.01, or 95:05 to about 99.9:0.01.

In some embodiments, the compound of Formula (III) is a compound of Formula (IIIa), (IIIb), (IIIc), or (IIId):

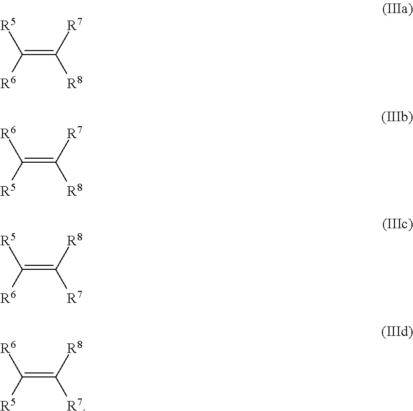

In some embodiments, the composition further comprises a solvent component. In some embodiments, the solvent component is water, acetonitrile, tetrahydrofuran, diethyl ether, methyl-t-butyl ether, dimethyoxyethane, bis(2-methoxyethyl) ether, or benzene substituted with 1, 2, 3, or 4 independently selected C₁₋₄ alkyl groups. In some embodiments, the solvent component is acetonitrile or benzene substituted with 1, 2, 3, or 4 independently selected C₁₋₄ alkyl groups. In some embodiments, the solvent component is acetonitrile or benzene substituted with 1 or 2 independently selected C₁₋₄ alkyl groups. In some embodiments, the solvent component is acetonitrile or benzene substituted with 1 or 2 independently selected methyl groups. In some embodiments, the solvent component is water, acetonitrile, toluene, o-xylene, m-xylene, p-xylene, or a mixture thereof In some embodiments, the solvent component is acetonitrile. In some embodiments, the solvent component a mixture of acetonitrile and water. In some embodiments, the solvent component is toluene. In some embodiments, the solvent component a mixture of toluene and water. In some embodiments, the solvent component is o-xylene, m-xylene, p-xylene, or a mixture thereof. In some embodiments, the solvent component is o-xylene, m-xylene, p-xylene, or a mixture thereof; and water.

In some embodiments, the composition further comprises a hypohalite salt component. In some embodiments, the hypohalite salt component is an alkali metal hypohalite salt or an alkali earth metal hypohalite salt. In some embodiments, the hypohalite salt component is an alkali metal hypohalite salt. In some embodiments, the hypohalite salt component is a hypochlorite salt. In some embodiments, the hypohalite salt component is selected from NaOCl, KOCl, NaOBr, and Ca(OCl)₂. In some embodiments, the hypohalite salt component is NaOCl.

In some embodiments, the composition further comprises a cationic phase transfer catalyst component. In some embodiments, the cationic phase transfer catalyst component is a quaternary ammonium salt. In some embodiments, the cationic phase transfer catalyst component is a quaternary phosphonium salt. In some embodiments, the cationic phase transfer catalyst component is (n-butyl)₄P⁺Br⁻, (n-butyl)₄N⁺Br⁻, (n-butyl)₄N⁺Br⁻, (n-butyl)₄N⁺HSO₄⁻, Ph₄P⁺Br⁻, (ethyl)₄N⁺Br⁻, or Aliquat®336.

In some embodiments, the composition comprises a compound of Formula (I):

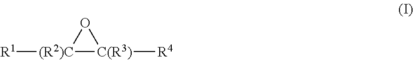

a compound of Formula (III):

R⁵—C(R⁶)═C(R⁷)—R⁸ (III)

and one or more of:

a) a solvent component;

b) a hypohalite salt component;

c) a cationic phase transfer catalyst component,

wherein the solvent component, hypohalite salt component, and ionic phase transfer catalyst components are as defined above.

In some embodiments, the composition comprises:
a compound of Formula (I) which is 2-fluoro-3-(trifluoromethyl)oxirane and a compound of Formula (III) which is 1,3,3,3-tetrafluoroprop-1-ene; or
a compound of Formula (I) which is 2,3-bis(trifluoromethyl)oxirane and a compound of Formula (III) which is 1,1,1,4,4,4-hexafluorobut-2-ene; or
a compound of Formula (I) which is 2-(trifluoromethyl)-3-(perfluoroethyl)oxirane and a compound of Formula (III) which is 1,1,1,4,4,5,5,5-octafluoropent-2-ene; or
a compound of Formula (I) which is 2-(trifluoromethyl)-3-(perfluoropropan-2-yl)oxirane and a compound of Formula (III) which is 1,1,1,5,5,5-hexafluoro-4,4-bis(trifluoromethyl)pent-2-ene; or
a compound of Formula (I) which is 2,3-bis(perfluoropropyl)oxirane and a compound of Formula (III) which is 1,1,1,2,2,3,3,6,6,7,7,8,8,8-tetradecafluorooct-4-ene; or
a compound of Formula (I) which is 2-(perfluorobutyl)-3-(perfluoroethyl)oxirane and a compound of Formula (III) which is 1,1,1,2,2,5,5,6,6,7,7,8,8,8-tetradecafluorooct-3-ene; or
a compound of Formula (I) which is 2,3-bis(perfluorobutyl)oxirane and a compound of Formula (III) which is 1,1,1,2,2,3,3,4,4,7,7,8,8,9,9,10,10,10-octadecafluorodec-5-ene; or
a compound of Formula (I) which is 2-(2,2,2-Trifluoroethoxy)-3-fluoro-2-(trifluoromethyl)-3-(perfluoroethyl)oxirane and a compound of Formula (III) which is 2-(2,2,2-trifluoroethoxy)-1,1,1,4,4,5,5,5-octafluoropent-2-ene; or
a compound of Formula (I) which is 2,3-dichloro-2,3-bis(trifluoromethyl)oxirane and a compound of Formula (III) which is 2,3-dichloro-1,1,1,4,4,4-hexafluorobut-2-ene; or
a compound of Formula (I) which is 2-fluoro-3-(perfluoropropan-2-yl)oxirane and a compound of Formula (III) which is 1,3,4,4,4-pentafluoro-3-(trifluoromethyl)but-1-ene; or
a compound of Formula (I) which is 2,3-difluoro-2-(perfluoroethyl)-3-(perfluoropropyl)oxirane and a compound of Formula (III) which is perfluoroheptene-3; or
a compound of Formula (I) which is 2,3-difluoro-2-(trifluoromethyl)-3-(perfluoropentyl)oxirane and a compound of Formula (III) which is perfluoroctene-2; or
a compound of Formula (I) which is 2,2,3,3,4,4-hexafluoro-6-oxa-bicyclo[3.1.0]hexane and a compound of Formula (III) which is 3,3,4,4,5,5-hexafluorocyclopent-1-ene; or
a compound of Formula (I) which is 2,2,3,3-tetrafluoro-5-oxabicyclo[2.1.0]pentane and a compound of Formula (III) which is 3,3,4,4-tetrafluorocyclobut-l-ene.

In some embodiments, each of the preceding compositions is a mixture of the trans-isomer of the compound of Formula (I) and the trans-isomer of the compound of Formula (III). In some embodiments, each of the preceding compositions is a mixture of the cis-isomer of the compound of Formula (I) and the cis-isomer of the compound of Formula (III).

In some embodiments, the composition consists essentially of the compound of Formula (I).

In some embodiments, the composition consists essentially of the compound of Formula (I) and the compound of Formula (III).

In some embodiments, the composition provided herein is prepared according to a process provided herein.

### Compounds

The present application further provides any of the compounds described in the process embodiments above, or in the Examples. Accordingly, the present application provides a compound of Formula (I):

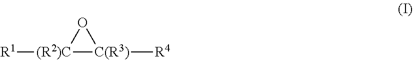

wherein:
R¹ and R⁴ are each independently H, Cl, F, Br, I, a partially fluorinated C₁₋₄ alkoxy, or a perfluorinated C₁₋₄ alkoxy;
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
   wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring.

In some embodiments:
R¹ and R⁴ are each independently H, Cl, F, a partially fluorinated C₁₋₄ alkoxy, or a perfluorinated C₁₋₄ alkoxy; and
R² and R³ are each independently selected from partially fluorinated or perfluorinated C₁₋₁₀ alkyl;
or alternatively, R² and R³ are each independently selected from partially fluorinated or perfluorinated C₁₋₅ alkylene, which together form a monocyclic ring.

In some embodiments, the compound of Formula (I) is the cis-isomer. In some embodiments, the compound of Formula (I) is the trans-isomer.

In some embodiments, the compounds are non-cyclic. Accordingly, in some embodiments, R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₁₀ alkyl.

In some embodiments of the non-cyclic compounds, R¹ and R⁴ are identical. In some embodiments of the non-cyclic compounds, R¹ and R⁴ are different. In some embodiments of the non-cyclic compounds, R¹ and R⁴ are each H. In some embodiments of the non-cyclic compounds, R¹ and R⁴ are each F. In some embodiments of the non-cyclic compounds, R¹ and R⁴ are each Cl. In some embodiments of the non-cyclic compounds, le is a partially fluorinated C₁₋₄ alkoxy and R⁴ is H.

In some embodiments of the non-cyclic compounds, R² and R³ are identical. In some embodiments of the non-cyclic compounds, R² and R³ are different. In some embodiments of the non-cyclic compounds, R² and R³ are each independently selected from partially fluorinated or perfluorinated C₁₋₁₀ alkyl. In some embodiments of the non-cyclic compounds, R² and R³ are each an independently selected perfluorinated C₁₋₁₀ alkyl. In some embodiments of the non-cyclic compounds, R² and R³ are each an independently selected partially fluorinated C₁₋₁₀ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated or perfluorinated C₁₋₆ alkyl. In some embodiments of the non-cyclic compounds, R² and R³ are each independently selected from partially fluorinated C₁₋₆ alkyl. In some embodiments of the non-cyclic compounds, R² and R³ are each independently selected from perfluorinated C₁₋₆ alkyl. In some embodiments of the non-cyclic compounds, R² and R³ are each independently CF₃, CF₂CF₃, CF(CF₃)₂, CF₂CF₂CF₃, CF₂CF₂CF₂CF₃, or CF₂CF₂CF₂CF₂CF_{3.}

In some embodiments, the compounds are cyclic. Accordingly, in some embodiments, R¹ and R⁴ are each independently H, Cl, F, a partially fluorinated C₁₋₄ alkoxy, or a perfluorinated C₁₋₄ alkoxy; and R² and R³ are each independently selected from partially fluorinated or perfluorinated C₁₋₅ alkylene, which together form a monocyclic ring.

In some embodiments of the cyclic compounds, R¹ and R⁴ are identical. In some embodiments of the cyclic compounds, R¹ and R⁴ are different. In some embodiments of the cyclic compounds, R¹ and R⁴ are each H. In some embodiments of the cyclic compounds, R¹ and R⁴ are each F. In some embodiments of the cyclic compounds, R¹ and R⁴ are each Cl. In some embodiments of the cyclic compounds, R¹ is a partially fluorinated C₁₋₄ alkoxy and R⁴ is H.

In some embodiments, R² and R³ are each independently selected from partially fluorinated or perfluorinated C₁₋₅ alkylene, which together form a monocyclic ring. In some embodiments, R² and R³ are each independently selected from a perfluorinated C₁₋₅ alkylene, which together form a monocyclic ring. In some embodiments, R² and R³, together with the carbon atoms to which they are attached, form a 4-6 membered monocyclic ring. In some embodiments, R¹ and R⁴ are each independently H or F; and R² and R³ are each independently selected from partially fluorinated or perfluorinated C₁₋₅ alkylene, which together form a monocyclic ring. In some embodiments, R¹ and R⁴ are each independently H or F; and R² and R³ are each an independently selected perfluorinated C₁₋₅ alkylene, which together form a monocyclic ring. In some embodiments, R¹ and R⁴ are each independently H or F; and R² and R³ are each independently selected from partially fluorinated or perfluorinated C₁₋₁₀ alkyl.

In some embodiments, the compound of Formula (I) is selected from the group consisting of:
2,3-difluoro-2-(trifluoromethyl)oxirane;
2-fluoro-3-(trifluoromethyl)oxirane;
2,3-bis(trifluoromethyl)oxirane;
2-(trifluoromethyl)-3-(perfluoroethyl)oxirane;
2-(trifluoromethyl)-3-(perfluoropropan-2-yl)oxirane;
2,3-bis(perfluoropropyl)oxirane;
2-(perfluorobutyl)-3-(perfluoroethyl)oxirane;
2,3-bis(perfluorobutyl)oxirane;
2-(2,2,2-Trifluoroethoxy)-3-fluoro-2-(trifluoromethyl)-3-(perfluoroethyl)oxirane;
2,3-dichloro-2,3-bis(trifluoromethyl)oxirane;
2-fluoro-3-(perfluoropropan-2-yl)oxirane;
2,2,3,3,4,4-hexafluoro-6-oxa-bicyclo[3.1.0]hexane;
2,2,3,3-tetrafluoro-5-oxabicyclo[2. 1 .0]pentane;
2,3-difluoro-2-(perfluoroethyl)-3-(perfluoropropyl)oxirane; and
2,3-difluoro-2-(trifluoromethyl)-3-(perfluoropentyl)oxirane.

In some embodiments, the compound of Formula (I) is selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-Hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

The compounds of Formula (I) provided herein include stereoisomers of the compounds. All stereoisomers, such as enantiomers and diastereomers, are intended unless otherwise indicated.

Trans- and cis-geometric isomers of the compounds described are described and may be isolated as a mixture of isomers or as separated isomeric forms. In some embodiments, the compound of Formula (I) is in the trans-configuration. In some embodiments, the compound of Formula (I) is in the cis-configuration. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Further, the process described herein results in the preservation of cis- or trans-stereochemistry.

Resolution of racemic mixtures of compounds can be carried out by any of numerous methods known in the art. For example, resolution of racemic mixtures can also be carried out by elution on a column packed with an optically active resolving agent (e.g., dinitrobenzoylphenylglycine). Suitable elution solvent composition can be determined by one skilled in the art.

The compounds of the invention can be found together with other substances, for example, water, solvents, salts, or olefins provided herein, or can be isolated.

### Methods of Use

The inventive compounds and compositions may be used in a wide range of applications, including their use as refrigerants, uses in high-temperature heat pumps, organic Rankine cycles, as fire extinguishing/fire suppression agents, propellants, foam blowing agents, solvents, and/or cleaning fluids.

### Refrigerants

The inventive compounds and compositions can act as a working fluid used to carry heat from a heat source to a heat sink. Such heat transfer compounds and compositions may also be useful as a refrigerant in a cycle wherein the fluid undergoes a phase change; that is, from a liquid to a gas and back, or vice versa. Examples of heat transfer systems include but are not limited to air conditioners, freezers, refrigerators, heat pumps, water chillers, flooded evaporator chillers, direct expansion chillers, walk-in coolers, mobile refrigerators, mobile air conditioning units and combinations thereof.

For example, the compounds and compositions provided herein may be useful in methods for producing cooling comprising evaporating any of the present compounds or compositions in the vicinity of a body to be cooled, and thereafter condensing said composition. Alternatively, the compounds and compositions provided herein may be useful in methods method for producing heating comprising condensing any of the present compounds or compositions in the vicinity of a body to be heated, and thereafter evaporating said compositions.

The compounds or compositions disclosed herein may also be useful as a replacement for a currently used (i.e., “incumbent”) refrigerant, including but not limited to R-123 (or HFC-123, 2,2-dichloro-1,1,1-trifluoroethane), R-11 (or CFC-11, trichlorofluoromethane), R-12 (or CFC-12, dichlorodifluoromethane), R-22 (chlorodifluoromethane), R-245fa (or HFC-245fa, 1,1,1,3,3-pentafluoropropane), R-114 (or CFC-114, 1,2-dichloro-1,1,2,2-tetrafluoroethane), R-236fa (or HFC-236fa, 1,1,1,3,3,3-hexafluoropropane), R-236ea (or HFC-236ea, 1,1,1,2,3,3-hexafluoropropane), R-124 (or HCFC-124, 2-chloro-1,1,1,2-tetrafluoroethane), among others.

Often, replacement refrigerants are most useful if capable of being used in the original refrigeration equipment designed for a different refrigerant, e.g., with minimal to no system modifications. In some embodiments, the compounds and compositions provided herein are useful as refrigerants and provide at least comparable cooling performance (meaning cooling capacity) as the refrigerant for which a replacement is being sought.

Examples of refrigeration systems the disclosed compositions may be useful in are equipment including commercial, industrial or residential refrigerators and freezers, ice machines, self-contained coolers and freezers, flooded evaporator chillers, direct expansion chillers, walk-in and reach-in coolers and freezers, and combination systems. In some embodiments, the disclosed compositions may be used in supermarket refrigeration systems. Additionally, stationary applications may utilize a secondary loop system that uses a primary refrigerant to produce cooling in one location that is transferred to a remote location via a secondary heat transfer fluid.

The compounds and compositions of the present invention may also have zero ozone depletion potential and low global warming potential (GWP). Additionally, the compounds and compositions of the present invention may have global warming potentials that are less than many hydrofluorocarbon refrigerants currently in use. Therefore, in accordance with the present invention, the compounds and compositions described herein may be useful in methods for producing cooling, producing heating, and transferring heat.

### High Temperature Heat Pumps

The compounds and compositions of the present invention may also be useful in methods for producing heating in a high temperature heat pump having a heat exchanger. In some embodiments, the method comprises extracting heat from a working fluid, thereby producing a cooled working fluid wherein said working fluid comprises a compound or composition provided herein.

Of note are high temperature heat pumps that may be used to heat air, water, another heat transfer medium or some portion of an industrial process, such as a piece of equipment, storage area or process stream. These high temperature heat pumps can produce maximum condenser operating temperatures greater than about 55° C. The maximum condenser operating temperature that can be achieved in a high temperature heat pump depends on the working fluid used. This maximum condenser operating temperature is limited by the normal boiling characteristics of the working fluid and also by the pressure to which the heat pump's compressor can raise the vapor working fluid pressure. This maximum pressure is also related to the working fluid used in the heat pump.

Also of note are heat pumps that are used to produce heating and cooling simultaneously. For example, a single heat pump unit may produce hot water for domestic use and may also produce cooling for comfort air conditioning in the summer.

The compounds and compositions described herein may also enable the design and operation of dynamic (e.g. centrifugal) or positive displacement (e.g. screw or scroll) heat pumps for upgrading heat available at low temperatures to meet demands for heating at higher temperatures. The available low temperature heat is supplied to the evaporator and the high temperature heat is extracted at the condenser or working fluid cooler (in a supercritical or transcritical mode). For example, waste heat can be available to be supplied to the evaporator of a heat pump operating at 25° C. at a location (e.g. a hospital) where heat from the condenser, operating at 85° C., can be used to heat water (e.g. for hydronic space heating or other service).

Some high temperature heat pumps operated with the compositions provided herein as the working fluid have vapor pressures below the threshold necessitating compliance with provisions of the ASME Boiler and Pressure Vessel Code. Such compositions are desirable for use in high temperature heat pumps. Of note are compositions where the working fluid consists essentially of from about 1 to about 100 weight percent of the fluorinated and perfluorinated epoxides provided herein (e.g., compounds of Formula (I)).

The compounds and compositions provided herein may meet the need for a non-flammable high temperature heat pump working fluid with reduced GWP and may therefore be useful as working fluids in high temperature heat pumps.

### Organic Rankine Cycles

The compounds and compositions provided herein may also be useful in processes for converting heat to mechanical work in a power cycle (e.g., an organic Rankine cycle). The power cycle includes the steps of heating a working fluid with a heat source to a temperature sufficient to pressurize the working fluid and causing the pressurized working fluid to perform mechanical work. In some embodiments, the process may utilize a sub-critical power cycle, trans-critical power cycle, or a super-critical power cycle.

An Organic Rankine Cycle (ORC) system is named for its use of organic working fluids that enable such a system to capture heat from low temperature heat sources such as geothermal heat, biomass combustors, industrial waste heat, and the like. The captured heat maybe converted by the ORC system into mechanical work and/or electricity. Organic working fluids are selected for their liquid-vapor phase change characteristics, such as having a lower boiling temperature than water.

The compounds and compositions provided herein may also be useful in processes of using a working fluid to convert heat to mechanical work by using a sub-critical power cycle. The ORC system is operating in a sub-critical cycle when the working fluid receives heat at a pressure lower than the critical pressure of the working fluid and the working fluid remains below its critical pressure throughout the entire cycle. This process comprises the following steps: (a) compressing a liquid working fluid to a pressure below its critical pressure; (b) heating the compressed liquid working fluid from step (a) using heat supplied by the heat source to form a vapor working fluid; (c) expanding the vapor working fluid from step (b) in an expansion device to generate mechanical work; (d) cooling the expanded working fluid from step (c) to form a cooled liquid working fluid; and (e) cycling the cooled liquid working fluid from step (d) to step (a) to repeat the cycle.

The compounds and compositions provided herein may also be useful in processes of using a working fluid to convert heat energy to mechanical work by using a trans-critical power cycle. The ORC system is operating as a trans-critical cycle when the working fluid receives heat at a pressure higher than the critical pressure of the working fluid. In a trans-critical cycle, the working fluid does not remain at a pressure higher than its critical pressure throughout the entire cycle. This process comprises the following steps: (a) compressing a liquid working fluid to a pressure above the working fluid's critical pressure; (b) heating the compressed working fluid from step (a) using heat supplied by the heat source; (c) expanding the heated working fluid from step (b) to lower the pressure of the working fluid below its critical pressure to generate mechanical work; (d) cooling the expanded working fluid from step (c) to form a cooled liquid working fluid; and (e) cycling the cooled liquid working fluid from step (d) to step (a) to repeat the cycle.

Typically for a trans-critical ORC, the temperature to which the working fluid is heated using heat from the heat source is in the range of from about 195° C. to about 300° C., preferably from about 200° C. to about 250° C., more preferably from about 200° C. to 225° C. Typical expander inlet pressures for trans-critical cycles are within the range of from about the critical pressure, 1.79 MPa, to about 7 MPa, preferably from about the critical pressure to about 5 MPa, and more preferably from about the critical pressure to about 3 MPa. Typical expander outlet pressures for trans-critical cycles are comparable to those for subcritical cycles.

The compounds and compositions provided herein may also be useful in processes of using a working fluid to convert heat energy to mechanical work by using a super-critical power cycle. An ORC system is operating as a super-critical cycle when the working fluid used in the cycle is at pressures higher than its critical pressure throughout the cycle. The working fluid of a super-critical ORC does not pass through a distinct vapor-liquid two-phase transition as in a sub-critical or trans-critical ORC. This method comprises the following steps: (a) compressing a working fluid from a pressure above its critical pressure to a higher pressure; (b) heating the compressed working fluid from step (a) using heat supplied by the heat source; (c) expanding the heated working fluid from step (b) to lower the pressure of the working fluid to a pressure above its critical pressure and generate mechanical work; (d) cooling the expanded working fluid from step (c) to form a cooled working fluid above its critical pressure; and (e) cycling the cooled working fluid from step (d) to step (a) for compression.

Typically for super-critical cycles, the temperature to which the working fluid is heated using heat from the heat source is in the range of from about 190° C. to about 300° C., preferably from about 200° C. to about 250° C., more preferably from about 200° C. to 225° C. The pressure of the working fluid in the expander is reduced from the expander inlet pressure to the expander outlet pressure. Typical expander inlet pressures for super-critical cycles are within the range of from about 2 MPa to about 7 MPa, preferably from about 2 MPa to about 5 MPa, and more preferably from about 3 MPa to about 4 MPa. Typical expander outlet pressures for super-critical cycles are within about 0.01 MPa above the critical pressure.

The compounds and compositions of the present invention may also be useful in ORC systems to generate mechanical work from heat extracted or received from relatively low temperature heat sources such as low pressure steam, industrial waste heat, solar energy, geothermal hot water, low-pressure geothermal steam (primary or secondary arrangements), or distributed power generation equipment utilizing fuel cells or prime movers such as turbines, micro-turbines, or internal combustion engines. One source of low-pressure steam could be the system known as a binary geothermal Rankine cycle. Large quantities of low-pressure steam can be found in numerous locations, such as in fossil fuel powered electrical generating power plants.

### Fire Extinguishing/Fire Suppression

The compounds and compositions provided herein may also be useful as fire extinguishing agents (either alone or in admixture with each other or in blends with other fire extinguishing agents) for use in methods of fire extinguishing or fire suppression. The other agents with which the compounds and compositions of this invention may be blended include, but are not limited to, chlorine and/or bromine containing compounds such as Halon 1301 (CF₃Br), Halon 1211 (CF₂BrCl), Halon 2402 (CF₂BrCF₂Br), Halon 251 (CF₃CF₂Cl) and CF₃CHFBr.

Thus, the compounds and compositions provided herein may be used in a total flooding fire extinguishing system in which the compound or composition (i.e., “agent”) is introduced to an enclosed region (e.g., a room or other enclosure) surrounding a fire at a concentration sufficient to extinguish the fire. In accordance with a total flooding system apparatus, equipment or even rooms or enclosures may be provided with a source of agent and appropriate piping, valves, and controls so as automatically and/or manually to be introduced at appropriate concentrations in the event that fire should break out. Thus, as is known to those skilled in the art, the fire extinguishant may be pressurized with nitrogen or other inert gas at up to about 600 psig at ambient conditions.

Alternatively, the compounds and compositions of the present invention may be applied to a fire through the use of conventional portable fire extinguishing equipment. It is usual to increase the pressure in portable fire extinguishers with nitrogen or other inert gasses in order to insure that the agent is completely expelled from the the extinguisher. Hydrofluorocarbon containing systems in accordance with this invention may be conveniently pressurized at any desirable pressure up to about 600 psig at ambient conditions.

In addition, the compounds and compositions provided herein may be useful in methods of suppressing a flame, said methods comprising contacting a flame with a fluid comprising a compound or composition of the present application. Any suitable methods for contacting the flame with the present composition may be used. For example, the compound or composition may be sprayed, poured, and the like, onto the flame, or at least a portion of the flame may be immersed in the flame suppression composition. In light of the teachings herein, those of skill in the art will be readily able to adapt a variety of conventional apparatus and methods of flame suppression for use in the present disclosure.

As will be appreciated by one of skill in the art, there are four general types of halocarbon fire and explosion protection applications:
1) In total-flood fire extinguishment and/or suppression applications, the agent is discharged into a space to achieve a concentration sufficient to extinguish or suppress an existing fire. Total flooding use includes protection of enclosed, potentially occupied spaces such, as computer rooms as well as specialized, often unoccupied spaces such as aircraft engine nacelles and engine compartments in vehicles.
2) In streaming applications, the agent is applied directly onto a fire or into the region of a fire. This is usually accomplished using manually operated wheeled or portable units. A second method, included as a streaming application, uses a “localized” system, which discharges the agent toward a fire from one or more fixed nozzles. Localized systems may be activated either manually or automatically.
3) In explosion suppression, an inventive composition of the present disclosure is discharged to suppress an explosion that has already been initiated. The term “suppression” is normally used in this application because the explosion is usually self-limiting. However, the use of this term does not necessarily imply that the explosion is not extinguished by the agent. In this application, a detector is usually used to detect an expanding fireball from an explosion, and the agent is discharged rapidly to suppress the explosion. Explosion suppression is used primarily, but not solely, in defense applications.
4) In inertion, an inventive composition of the present disclosure is discharged into a space to prevent an explosion or a fire from being initiated. Often, a system similar or identical to that used for total-flood fire extinguishment or suppression is used. Usually, the presence of a dangerous condition (for example, dangerous concentrations of flammable or explosive gases) is detected, and the inventive composition of the present disclosure is then discharged to prevent the explosion or fire from occurring until the condition can be remedied.

Preferably, the extinguishing process involves introducing the compounds and compositions of the present disclosure to a fire or flame in an amount sufficient to extinguish the fire or flame. One skilled in this field will recognize that the amount of flame suppressant needed to extinguish a particular fire will depend upon the nature and extent of the hazard. When the flame suppressant is to be introduced by flooding, cup burner test data are useful in determining the amount or concentration of flame suppressant required to extinguish a particular type and size of fire.

Laboratory tests useful for determining effective concentration ranges of an inventive composition when used in conjunction with extinguishing or suppressing a fire in a total-flood application or fire inertion are described, for example, in U.S. Pat. No. 5,759,430.

### Propellants

The compounds and compositions provided herein may also be useful as propellants, e.g., in a sprayable composition. The active ingredient to be sprayed together with inert ingredients, solvents, and other materials may also be present in a sprayable composition. Preferably, the sprayable composition is an aerosol. Suitable active materials to be sprayed include, but are not limited to, cosmetic materials, such as deodorants, perfumes, hair sprays, cleaners, and polishing agents as well as medicinal materials such as anti-asthma and anti-halitosis medications.

The present invention further relates to a process for producing aerosol products comprising the step of adding a compound or composition as described herein to active ingredients in an aerosol container, wherein said compound or composition functions as a propellant.

In some embodiments, the compounds and compositions provided herein may be useful as a co-propellant in a sprayable composition. In some embodiments, the present application provides a method of spraying an active material, comprising spraying a composition comprising a propellant component and a co-propellant component, wherein the co-propellant component is a compound of Formula (I) as described herein, or a mixture of compounds of Formula (I).

### Foam Blowing Agents

The compounds and compositions provided herein may also be useful as foam blowing agents (either alone or in combination with other agents), for example, in foamable compositions. The foamable composition is preferably a thermoset or thermoplastic foam composition, prepared using the compounds or compositions of the present disclosure. In such foam embodiments, one or more of the present compounds or compositions are included as or part of a blowing agent in a foamable composition, wherein the foamable composition preferably includes one or more additional components capable of reacting and/or foaming under the proper conditions to form a foam or cellular structure.

Closed-cell polyisocyanate-based foams are widely used for insulation purposes, for example, in building construction and in the manufacture of energy efficient electrical appliances. In the construction industry, polyurethane (polyisocyanurate) board stock is used in roofing and siding for its insulation and load-carrying capabilities. Poured and sprayed polyurethane foams are widely used for a variety of applications including insulating roofs, insulating large structures such as storage tanks, insulating appliances such as refrigerators and freezers, insulating refrigerated trucks and railcars, etc.

A second type of insulating foam is thermoplastic foam, primarily polystyrene foam. Polyolefin foams (e.g., polystyrene, polyethylene, and polypropylene) are widely used in insulation and packaging applications. These thermoplastic foams were generally made with CFC-12 (dichlorodifluoromethane) as the blowing agent. More recently HCFCs (HCFC-22, chlorodifluoromethane) or blends of HCFCs (HCFC-22/HCFC-142b) or HFCs (HFC-152a) have been employed as blowing agents for polystyrene.

A third type of insulating foam is phenolic foam. These foams, which have attractive flammability characteristics, have been generally made with CFC-11 (trichlorofluoromethane) and CFC-113 (1,1,2-trichloro-1,2,2-trifluoroethane) blowing agents.

In addition to closed-cell foams, open-cell foams are also of commercial interest, for example in the production of fluid-absorbent articles. U.S. Pat. No. 6,703,431 (Dietzen, et. al.) describes open-cell foams based on thermoplastics polymers that are useful for fluid-absorbent hygiene articles such as wound contact materials. U.S. Pat. No. 6,071,580 (Bland, et. al.) describes absorbent extruded thermoplastic foams which can be employed in various absorbency applications. Open-cell foams have also found application in evacuated or vacuum panel technologies, for example in the production of evacuated insulation panels as described in U.S. Pat. No. 5,977,271 (Malone). Using open-cell foams in evacuated insulation panels, it has been possible to obtain R-values of 10 to 15 per inch of thickness depending upon the evacuation or vacuum level, polymer type, cell size, density, and open cell content of the foam. These open-cell foams have traditionally been produced employing CFCs, HCFCs, or more recently, HFCs as blowing agents.

Multimodal foams are also of commercial interest, and are described, for example, in U.S. Pat. Nos. 6,787,580 (Chonde, et. al.) and 5,332,761 (Paquet, et. al.). A multimodal foam is a foam having a multimodal cell size distribution, and such foams have particular utility in thermally insulating articles since they often have higher insulating values (R-values) than analogous foams having a generally uniform cell size distribution. These foams have been produced employing CFCs, HCFCs, and, more recently, HFCs as the blowing agent.

All of these various types of foams require blowing (i.e., expansion) agents for their manufacture. Insulating foams depend on the use of halocarbon blowing agents, not only to foam the polymer, but primarily for their low vapor thermal conductivity, a very important characteristic for insulation value.

The methods of forming a foam generally comprise providing a blowing agent composition of the present disclosure, adding (e.g., directly or indirectly) the blowing agent composition to a foamable composition, and reacting the foamable composition under the conditions effective to form a foam or cellular structure. Any of the methods well known in the art, such as those described in “Polyurethanes Chemistry and Technology,” Volumes I and II, Saunders and Frisch, 1962, John Wiley and Sons, New York, N.Y., which is incorporated herein by reference, may be used or adapted for use in accordance with the foam embodiments.

Representative foamed products that can be made in accordance with the present disclosure include, for example: (1) polystyrene foam sheet for the production of disposable thermoformed packaging materials; e.g., as disclosed in York, U.S. Pat. No. 5,204,169; (2) extruded polystyrene foam boards for use as residential and industrial sheathing and roofing materials, which may be from about 0.5 to 6 inches (1.25 to 15 cm) thick, up to 4 feet (122 cm) wide, with cross-sectional areas of from 0.17 to 3 square feet (0.016 to 0.28 square meter), and up to 27 feet (813 meters) long, with densities of from about 1.5 to 10 pounds per cubic foot (pcf) (25 to 160 kilograms per cubic meter (kg/m³); (3) expandable foams in the form of large billets which may be up to about 2 feet (61 cm) thick, often at least 1.5 feet 46 cm) thick, up to 4 feet (1.22 meters) wide, up to 16 feet (4.8 meters) long, having a cross-sectional area of about 2 to 8 square feet (0.19 to 0.74 square meter) and a density of from 6 to 15 pcf (96 to 240 kg/m³). Such foamed products are more fully described by Stockdopole and Welsh in the Encyclopedia of Polymer Science and Engineering, vol. 16, pages 193-205, John Wiley & Sons, 1989; hereby incorporated by reference.

### Solvents & Cleaning Fluids

The compounds and compositions provided herein may also be useful as inert media for polymerization reactions, fluids for removing particulates from metal surfaces, as carrier fluids that may be used, for example, to place a fine film of lubricant on metal or plastic parts, or as buffing abrasive agents to remove buffing abrasive compounds from polished surfaces such as metal. The compounds and compositions of the invention may also be used as displacement drying agents for removing water (i.e., “dewatering” agents), such as from jewelry or metal parts, as resist developers in conventional circuit manufacturing techniques including chlorine-type developing agents, or as strippers for photoresists when used with, for example, a chlorohydrocarbon such as 1,1,1-trichloroethane or trichloroethylene.

For example, the compounds and compositions provided herein may be useful in in dewatering processes, wherein water originally bound to the surface of the substrate is displaced by solvent and/or surfactant and leaves with the dewatering composition. Optionally, minimal amounts of surfactant remaining adhered to the substrate can be further removed by contacting the substrate with surfactant-free halocarbon solvent. Holding the article in the solvent vapor or refluxing solvent will further decrease the presence of surfactant remaining on the substrate. Removal of solvent adhering to the surface of the substrate is effected by evaporation. Evaporation of solvent at atmospheric or subatmospheric pressures can be employed and temperatures above and below the boiling point of the halocarbon solvent can be used.

Many industries use aqueous compositions for the surface treatment of metals, ceramics, glasses, and plastics. Cleaning, plating, and deposition of coatings are often carried out in aqueous media and are usually followed by a step in which residual water is removed. Hot air drying, centrifugal drying, and solvent-based water displacement are methods used to remove such residual water.

The primary function of the dewatering or drying solvent (e.g., compounds or compositions provided herein) in a dewatering or drying composition is to reduce the amount of water on the surface of a substrate being dried. The primary function of the surfactant is to displace any remaining water from the surface of the substrate. When the composition and surfactant are combined, a highly effective displacement drying composition is attained.

In some embodiments, the compounds and compositions provided herein may also be useful as solvents in fluorolubricant compositions. Fluorolubricants are widely used as lubricants in the magnetic disk drive industry to decrease the friction between the head and disk, that is, reduce the wear and therefore minimize the possibility of disk failure. Invariably, during normal disk drive application, the head and the disk surface will make contact. To reduce wear on the disk, from both sliding and flying contacts, it must be lubricated.

There is a need in the industry for improved methods for deposition of fluorolubricants. The use of certain solvents, such as CFC-113 and PFC-5060, has been regulated due to their impact on the environment. Therefore, solvents that will be used in this application should consider environmental impact. Also, such solvent must dissolve the fluorolubricant and form a substantially uniform or uniform coating of fluorolubricant. Additionally, existing solvents have been found to require higher fluorolubricant concentrations to produce a given thickness coating and produce irregularities in uniformity of the fluorolubricant coating.

Accordingly, the compounds and compositions provided herein may have utility as solvents, carrier fluids, dewatering agents, degreasing solvents, or defluxing solvents. It is desirable to identify new agents for these applications with reduced global warming potential.

### Gaseous Dielectrics

A dielectric gas, or insulating gas, is a dielectric material in gaseous state. Its main purpose is to prevent or rapidly quench electric discharges. Dielectric gases are used as electrical insulators in high voltage applications, e.g., transformers, circuit breakers, switchgear (namely high voltage switchgear), and radar waveguides. As used herein, the term “high voltage” shall be understood to mean above 1000 V for alternating current, and at least 1500 V for direct current. The inventive compounds and compositions can be useful as gaseous dielectrics in high voltage applications.

### EXAMPLE S

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

¹H, ¹³C, and ¹⁹F NMR sectra were recorded in CDCl₃ on a Varian VNMRS (499.87 MHz) instrument using CFCl₃ or TMS as internal reference standards. GC and GC/MS analyses were carried out on an HP-6890 instrument, using an HP FFAP capillary column and either TCD (GC) or mass- selective (GS/MS) detectors, respectively. Acetonitrile, tetrabutylammonium hydrogen sulfate, tetrabutylphosphium bromide Aliquat® 336, xylene, o-xylene were obtained from commercial source (Aldrich) and used without further purification. Commercially available sodium hypochlorite solution (typically 10-15% of available chlorine) was available from Sigma-Aldrich and was stored refrigerated. Purity of all isolated compounds was established to be at least 98-99% by GC and NMR spectroscopy (the remainder was determined to be remaining starting material or solvent unless specified otherwise. Epoxide of perfluoropentene-2 was identified based on reported NMR data and data of GC/MS (see e.g., Kolenko et al, Izv. Akad. Nauk. SSSR, Ser. Khim, 1979, 2509-2512).

### Example 1

### Cis-2-fluoro-3-(trifluoromethyl)oxirane

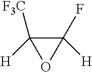

A 250 mL round bottom flask was equipped with a magnetic stir bar, a dry ice condenser, and a thermowell. The flask was charged with xylenes (30 mL, 0.24 moles), 15% w/w chilled sodium hypochlorite (100 mL, 1.49 moles), and Aliquat® 336 (5% mol, 1 mL). The reaction mixture was stirred at room temperature. An addition funnel containing olefin (Z)-1,3,3,3-tetrafluoroprop-1-ene, (87.71 mmoles, 10 grams) was fixed onto the reaction flask and olefin was added dropwise to the reaction mixture over a period of 30 minutes. A slight exotherm was observed once the internal temperature reached 25° C. The reaction mixture was sampled every hour over the first 6 hours and then stirred overnight. The contents of the flask were then transferred to a separatory funnel and the aqueous layer was discarded. The organic layer was dried with magnesium sulfate and then filtered into a clean round bottom flask. The filtrate was then subjected to reduced pressure; any remaining olefin and epoxide were collected in a dry ice cold trap and subsequently distilled to provide the desired product with b.p. 54-55° C.; isolated yield: 35%. ¹⁹F (CDCl₃): −68.88 (3F, dm, 12.5 Hz), −164.84 (1F, dqm, 84.0, 12.5, 3.5 Hz) ppm; ¹H NMR (CDCl₃): 3.28 (1H, m, 4.9, 3.0, 1.6 Hz), 5.61 (1H, d quint. 84.0, 1.8 Hz) ppm; ¹³C NMR {H}, (CDCl₃): 57.36 (qd, 42.6, 15.9 Hz), 85.56 (dq, 278.4, 1.5 Hz), 121.42 (qd, 275.4, 6.4 Hz) ppm MS (m/z): 130 (M⁺, C₃H₂F₄O⁺).

### Example 2

### Trans-2-fluoro-3-(trifluoromethyl)oxirane

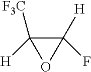

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (10% mol, 110 g). The flask was chilled to 0° C. An addition funnel containing olefin (E)-1,3,3,3-tetrafluoroprop-1-ene (3.36 moles, 383 grams) was fixed onto the reaction flask; olefin was added dropwise to the reaction mixture over a period of 60 minutes. An exotherm was initially observed but the controlled by an ice bath to maintain the internal temperature at no more than 15° C. The reaction mixture was sampled every hour over the first 3 hours and then stopped after this time. Once the reaction was complete, the contents of the flask were transferred to a separatory funnel and the aqueous layer was discarded. The organic layer was dried with magnesium sulfate and then filtered into a clean round bottom flask. The filtrate was then subjected to reduced pressure; any remaining olefin and epoxide were collected in a dry ice cold trap and subsequently distilled to provide the desired product with b.p. 19-20° C.; isolated yield: 37%. ¹⁹F NMR (CDCl₃): −73.19 (3F, d, 5.5 Hz), −155.92(1F, dm, 83.9, 1.4 Hz) ppm; ¹H NMR (CDCl₃): 3.67(1H, dq, 5.1, 2.8 Hz), 5.68(1H, d, 83.9 Hz) ppm; MS (m/z): 130(M⁺, C₃H₂F₄O⁺).

### Example 3

### Trans-2,3-bis(trifluoromethyl)oxirane

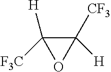 [SMILES: FC(F)(F)C1OC1C(F)(F)F]

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (7.5% mol, 85.3 g). The flask was chilled to 0° C. An addition funnel containing olefin (E)-1,1,1,4,4,4-hexafluorobut-2-ene (3.35 moles, 550 grams) was fixed onto the reaction flask; olefin was added dropwise to the reaction mixture over a period of 30 minutes. An exotherm was initially observed but the controlled by an ice bath to maintain the internal temperature at no more than 15° C. The reaction mixture was sampled every hour over the first 3 hours and then stopped after this time. Once the reaction was completed, the contents of the flask were transferred to a separatory funnel the organic layer was separated, dried over magnesium sulfate and filtered into a clean round bottom flask. The crude product was transferred into a dry ice cold trap under reduced pressure and subsequently distilled, to provide the desired product with b.p. 35° C.; isolated yield 80%. NMR data (CDCl₃): ¹⁹F: −74.38 (d, J=3.9 Hz) ppm; ¹H: 3.72 (m, not first order) ppm; ¹³C {H}: 50.38 (qq, J=43.15, 3.12 Hz), 120.91 (q, 275.8) ppm; M/Z: 180 M+ (C₄H₂F₆O).

### Example 4

### Cis-2,3-bis(trifluoromethyl)oxirane

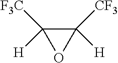 [SMILES: FC(F)(F)C1OC1C(F)(F)F]

A 5000 mL round bottom flask was equipped with a mechanical stir bar, a dry ice condenser, an addition funnel, and a thermowell. The flask was charged with xylenes (800 mL), 15% w/w chilled sodium hypochlorite (2400 mL), and tetrabutylphosphonium bromide (7.5% mol, 85.3 g). The flask was chilled to 0° C. An addition funnel containing olefin (Z)-1,1,1,4,4,4-hexafluorobut-2-ene (3.35 moles, 550 grams) was fixed onto the reaction flask; olefin was added dropwise to the reaction mixture over a period of 30 minutes. An exotherm was initially observed but the controlled by an ice bath to maintain the internal temperature at no more than 15° C. The reaction mixture was sampled every hour over the first 3 hours and then stopped after this time. Once the reaction was completed, the contents of the flask were transferred to a separatory funnel the organic layer was separated, dried over magnesium sulfate and filtered into a clean round bottom flask. The crude product was transferred into in a dry ice cold trap under reduced pressure and subsequently distilled, to provide the desired product with b.p. 64-65° C.; 425.8 g; isolated yield 70.5%. NMR data (CDCl₃): ¹⁹F: −67.69 (m) ppm; ¹H: 3.62 (m, not first order) ppm; ¹³C {H}: 52.71 (m), 120.08 (q, 276.2) ppm; MS (m/Z): 180 M⁺ (C₄H₂F₆O).

### Example 5

### Trans-2-(trifluoromethyl)-3-(perfluoroethyl)oxirane

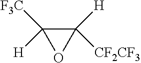

To a mixture of 40 mL of NaOCl, 10 mL of ACN, and 0.68 g of tetrabutylammonium hydrogensulfate was added 12 g of (E)-1,1,1,4,4,5,5,5-octafluoropent-2-ene and the reaction mixture was vigorously agitated at ambient temperature. The conversion of the olefin was 90% after 24 h. At this point the reaction mixture was diluted with water, and the organic layer was separated, dried over MgSO₄, and distilled to give 7 g (54%) of epoxide, >99% purity, b.p. 49-49.5° C. ¹⁹F NMR (CDCl₃): −74.10 (3F, d, 4.3 Hz), −84.10(3F, s), −127.03(1F, ddq, 275.7, 8.8, 1.4 Hz), −127.48(1F, dd, 275.7, 9.8) ppm; ¹H NMR (CDCl₃): 3.75 (m) ppm; ¹³C {H}NMR (CDCl₃): 49.29(q, 28.3, 3.0 Hz), 49.75(qdd, 43.2, 5.5, 3.4 Hz), 10.87(tq, 256.4, 38.4), 118.13(qt, 287.6, 53.0 Hz), 120.74(q, 278.6) ppm.

### Example 6

### Trans-2-(trifluoromethyl)-3-(perfluoropropan-2-yl)oxirane

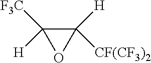

To a mixture of 200 mL of NaOCl, 30 mL of toluene, and 3.6 g of Aliquat® 336, 26.4 g (0.1 mol) of (E)-1,1,1,5,5,5-hexafluoro-4,4-bis(trifluoromethyl)pent-2-ene was added dropwise, and the internal temperature was maintained at 20-26° C. The reaction mixture was vigorously agitated for 20 h at ambient temperature at which time conversion of the olefin was determined to be >98%. The reaction mixture was transferred under dynamic vacuum (˜10mm Hg) into a cold trap (−78° C.), dried over MgSO₄, and distilled using a Vigroux column. Material with b.p. 70-71° C. (4.5 g) was isolated, which was found to be the epoxide containing 5% toluene, (NMR) and an additional 9 g of the epoxide (b.p. 72-90° C.) containing ˜20% toluene. Calculated yield of epoxide was 38%.

### Example 7

### Trans-2,3-bis(perfluoropropyl)oxirane

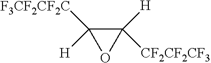

The title compound was prepared following the same protocol described in Example 6. Purity: 82% (18% toluene); calc. yield 71%. ¹⁹F NMR (CDCl₃): −80.96(3F, t, 8.5 Hz), −124.10(1F, d quint., 282, 9.1, Hz), −125.20(1F, q quint., 282, 9.0), −128.04(2F, d, 4.2 Hz) ppm; ¹H NMR (CDCl₃): 3.79 (1H, t, 9.0 Hz) ppm.

### Example 8

### Trans-2-(perfluorobutyl)-3-(perfluoroethyl)oxirane

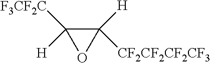

A mixture of 20 mL of sodium hypochlorite, 6.0 mL of o-xylene, 0.35 g tetrabutylphosphonium bromide (10 mol %), and 4.0 g of (E)-1,1,1,2,2,5,5,6,6,7,7,8,8,8-tetradecafluorooct-3-ene was agitated at ambient temperature for 24 h (conversion of olefin: 100%). The reaction mixture was then diluted with water and the organic layer was separated, washed by water, and dried over MgSO₄ and analyzed by NMR. Isolated material (9 g) was found to be a mixture of desired epoxide, o-xylene (ratio 35:65) and some tetrabutylphosphonium bromide (NMR). ¹⁹F NMR (CDCl₃): −81.27 (3F, tt, 9.9, 2.7 Hz), −84.38(3F,s), −122.99(1F, dq, 279.0, 9.8 Hz), −124.80(2F,m), −126.42(2F, m), −125.20(1F, q quint., 282, 9.0), −127.23 (1F, dd., 276.7, 10.5 Hz) −127.84 (1F, dd, 276.7, 9.0 Hz) ppm; ¹H NMR (CDCl₃): 3.73 (1H, t, 9.2 Hz), 3.79 (1H, t, 9.0 Hz) ppm

### Example 9

### Trans-2,3-bis(perfluorobutyl)oxirane

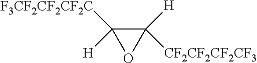

A mixture of 20 mL of sodium hypochlorite, 6.0 mL of o-xylene, 0.3 g tetrabutylphosphonium bromide (10 mol %), and 4.0 g of (E)-1,1,1,2,2,3,3,4,4,7,7,8,8,9,9,10,10,10-octadecafluorodec-5-ene was agitated at ambient temperature for 24 h (conversion of olefin was 90%). The reaction mixture was then diluted with water and the organic layer was separated, washed by water, and dried over MgSO₄ and analyzed by NMR. Isolated material (9.5 g) was found to be a mixture of desired epoxide, starting olefin, o-xylene (ratio 33.7:4.2:62.1) and some tetrabutylphosphonium bromide (NMR). ¹⁹F NMR (CDCl₃): −81.17 (3F, tt, 9.8, 2.7 Hz), −123.20 (1F, dq, 280.1, 12.7 Hz), −124.54 (1F, dq, 280.1, 10.2 Hz), −124.68 (2F, m), −124.68 (2F, m), −126.36 (2F, m) ppm; ¹H NMR (CDCl₃): 3.77 (t, 8.9 Hz) ppm

### Example 10

### 2-(2,2,2-Trifluoroethoxy)-3-fluoro-2-(trifluoromethyl)-3-(perfluoroethyl)oxirane (mixture of E and Z isomers)

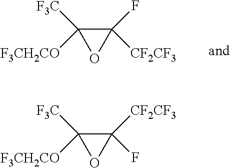

A mixture of 260 mL of sodium hypochlorite, 3.4 g tetrabutylammonium hydrogensulfate, and 60 g of 2-(2,2,2-trifluoroethoxy)-1,1,1,3,4,4,5,5,5-nonafluoropent-2-ene was agitated at ambient temperature for 24 h. The reaction mixture was then diluted with 500 mL of water and the organic layer was separated, washed by water, dried over MgSO₄, and distilled to afford 28 g of the desired epoxide (mixture of isomers), b.p. 91-94° C. (main 93° C., mixture of isomers, ratio˜95:5). Yield: 46%. Major isomer:

¹⁹F NMR (CDCl₃): −72.89(3F,d, 14.3 Hz), −75.08(3F,m), −82.88 (3F, t, 8.5 Hz), −124.10(1F, d quint., 282, 9.1, Hz), −124.83(1F, dt., 286.1, 3.7), −151.79(1F m), ppm; ¹H NMR (CDCl₃): 4.10 (1H, m), 4.24 (1H, m) ppm.

### Example 11

### 2,3-Dichloro-2,3-bis(trifluoromethyl)oxirane (mixture of isomers)

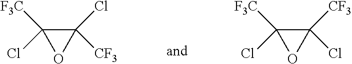 [SMILES: FC(F)(F)C1(Cl)OC1(Cl)C(F)(F)F ; FC(F)(F)C1(Cl)OC1(Cl)C(F)(F)F]

A 1000 mL round bottom flask was equipped with a magnetic stir bar, a dry ice condenser, and a thermowell. The flask was charged with acetonitrile (50 mL, 0.95 moles), 15% w/w chilled sodium hypochlorite (450 mL, 6.71 moles), and the tetrabutylammonium hydrogen sulfate (5 mol %, 6.0 grams). The reaction mixture was stirred at room temperature. An addition funnel containing (E/Z)-2,3-dichloro-1,1,1,4,4,4-hexafluorobut-2-ene (0.33 moles, 78 grams) was fixed onto the reaction flask; olefin was added dropwise to the reaction mixture over a period of 30 minutes. A slight exotherm was observed once the internal temperature reached 25° C. The reaction mixture stirred for 48 h. The contents of the flask were transferred to a separatory funnel and the organic layer was separated, dried over magnesium sulfate and filtered into a clean round bottom flask. The filtrate was then subjected to reduced pressure, crude product was collected in a dry ice cold trap and subsequently distilled to provide the desired product, with b.p. 68-69, (15.73 g, yield 18.9%).

### Examples 12A-12B

### Trans-2-fluoro-3-(perfluoropropan-2-yl)oxirane (Example 12A) and cis-2-fluoro-3-(perfluoropropan-2-yl)oxirane (Example 12B)

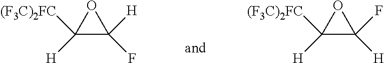

A 1000 mL round bottom flask was equipped with a magnetic stir bar, a dry ice condenser, and a thermowell. The flask was charged with xylenes or toluene (100 mL), 15% w/w chilled sodium hypochlorite (500 mL, 6.71 moles), and Aliquat® 336 (5 mol %, 5 grams). The reaction mixture was stirred at 0° C. for the entire reaction. An addition funnel containing olefin (trans or cis-isomers of 1,3,4,4,4-pentafluoro-3-(trifluoromethyl)but-1-ene, 0.24 moles, 52.6 grams) was fixed onto the reaction flask; olefin was added dropwise to the reaction mixture over a period of 30 minutes. A slight exotherm was observed once addition of the olefin was started. The reaction mixture was sampled every hour over the first 6 hours and then stirred overnight. After 24 h, the contents of the flask were transferred to a separatory funnel and the organic layer was separated, dried with magnesium sulfate and filtered into a clean round bottom flask. The crude product was transferred into cold trap (−78° C.) under reduced pressure and distilled at atmospheric pressure.

### Example 12A

(xylenes as a solvent): E-isomer, b.p. 55.5° C.; yield 47.8%. ¹⁹F NMR (CDCl₃): −75.60 (3F, quint, 8.8 Hz), −75.77(3F, quint., 8.7 Hz), −156.83 (1F, dd, 83.2, 2.8 Hz), −195.66 (1F, d, sept., 20.3, 8.1 Hz) ppm; ¹H NMR (CDCl₃): 3.67 (1H, dd, 20.3, 3.6 Hz), 5.64(d, 83.2 Hz) ppm.

### Example 12B

(xylenes as a solvent): E-isomer b.p. 88-89° C.; yield 17%, purity 80%, contained 20% of xylenes). ¹⁹F NMR (CDCl₃): −75.41 (3F, m), −76.26(3F, m), −163.20 (1F, dddq, 83.9, 42.9,5.5, 3.7 Hz), −195.41 (1F, d.d. quint., 42.9, 19.4, 3.7 Hz) ppm ¹H NMR (CDCl₃): 3.24 (1H, d, 19.4 Hz), 5.67 (1H, dm, 83.9, 1.0 Hz) ppm;

### Example 13

### 2,2,3,3,4,4-Hexafluoro-6-oxa-bicyclo[3.1.0]hexane

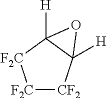 [SMILES: FC1(F)C2OC2C(F)(F)C1(F)F]

The reaction was performed using 120 mL of NaOCl, 30 mL of ACN, 2.5 g of tetrabutylammonium hydrogen sulfate (˜3 mol %), and 20 g (0.11 mol) of 3,3,4,4,5,5-hexafluorocyclopent-1-ene. The reaction was mildly exothermic reaction at 20-27° C. The reaction mixture was agitated at 20-25° C. for 4 h, then the organic phase was separated, washed by water, and dried over MgSO₄. The crude mixture was distilled to give 16 g of epoxide as single Z-isomer (NMR), b.p. 98-101° C. (main 100-101° C.), 98% purity, containing ˜2% of starting olefin, GC, NMR). Calculated yield: 75%. ¹⁹F (CDCl₃): −114.55 (1F, dm, 248.5 Hz), −115.22(2F, d, 262.2 Hz), −125.75(2F, dm, 263.2 Hz), −140.61(1F, dm, 248.5 Hz) ppm; ¹H NMR (CDCl₃): 3.99 (m) ppm; ¹³C {H}: 51.16 (m), 111.93 (m, two CF₂-groups); MS (m/z): 192 (M⁺, C₅H₂F₆O⁺).

### Example 14

### 2,2,3,3-Tetrafluoro-5-oxabicyclo[2.1.0]pentane

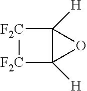

The starting material for this reaction, 3,3,4,4-tetrafluorocyclobut-1-ene, was prepared from 1-chloro-2,2,3,3-tetrafluorocyclobutane according to previously reported procedures (see e.g., Coffman et al, J. Am. Chem. Soc. 1949, 71:490-496). The title product was prepared using 120 mL of NaOCl, 30 mL of xylenes, 2.5 g of tetrabutylammonium hydrogen sulfate (˜3 mol %), and 30 g (0.24 mol) of 3,3,4,4-tetrafluorocyclobut-1-ene, which was added dropwise to the reaction mixture at 15-20° C. Mildly exothermic reaction at 20-27° C. was observed. The reaction mixture was agitated at 20-25° C. for 4 h (conversion of starting material 100%), then the organic phase was separated, washed by water, and dried over MgSO₄. Distillation of crude mixture gave 21 g (63%) of cis-epoxide as a single isomer (NMR), b.p. 73-74° C. (98% purity, containing ˜2% of xylenes, GC). ¹⁹F (CDCl₃): −117.77 (2F, dm, 204.0 Hz), −127.49(2F, dm, 204.0 Hz) ppm; ¹H NMR (CDCl₃): 4.38 (m) ppm; MS (m/z): 142 (M⁺, C₄H₂F₆O⁺);

### Example 15

### General Procedure for Preparation Epoxides of Perfluoroolefins

A 500 mL round bottom flask was equipped with a magnetic stir bar, a dry ice condenser, and a thermowell. The flask was charged with acetonitrile (25 mL, 0.47 moles), 15% w/w chilled sodium hypochlorite (250 mL, 3.72 moles), and the desired phase transfer catalyst (e.g., tetrabutylammonium hydrogen sulfate, 5 mol %, 3.06 g). The reaction mixture was stirred at room temperature. An addition funnel containing olefin (0.18 mole, typically a mixture of 18-90% of trans- and 10-20% cis-isomers) was fixed onto the reaction flask; olefin was added dropwise to the reaction mixture over a period of 30 minutes. A slight exotherm was observed once the internal temperature reached 25° C. The reaction temperature was maintained at <25° C. using a cooling bath. The reaction mixture was sampled every hour over the first 6 hours. Once the reaction was complete, the contents of the flask were transferred to a separatory funnel and organic layer was separated, washed with water, and dried over magnesium sulfate. The desired epoxides were isolated as a mixture of trans- and cis- isomers (determined by NMR), with ratios very similar to those observed in the corresponding starting material.

### Example 16

### 2,3-Difluoro-2-(perfluoroethyl)-3-(perfluoropropyl)oxirane (mixture of isomers)

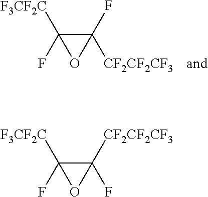

The title compound was prepared according to the general procedure described in Example 15, using perfluoroheptene-3 (98% purity, 2% of perfluoroheptene-2) as starting material. The epoxide (mixture trans- and cis- isomers 98:2) was isolated in 75% yield, b.p. 80° C.

### Example 17

### 2,3-Difluoro-2-(trifluoromethyl)-3-(perfluoropentyl)oxirane (mixture of isomers)

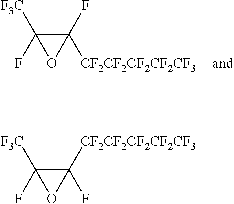

The title compound was prepared according to the general procedure described in Example 15, using perfluoroctene-2 as starting material. Purity of isolated epoxide was 95% (5% starting material); calc. yield: 84%; ratio trans-/cis-epoxides: 95:5.

### Example 18

### Comparative Example—Effect of Phase Transfer Catalyst on Conversion Perfluoroheptene-3 in Synthesis of 2,3-difluoro-2-(perfluoroethyl)-3-(perfluoropropyl)oxirane

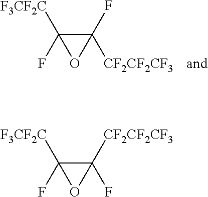

In 20 mL sample vial equipped with magnetic stir bar was added 15 mL of NaOCl solution and 3.5 g of perfluoroheptene-3 (98% purity) and 1 mL of acetonitrile. The sample vial was closed, placed on magnetic stir plate, the agitation speed was set to 1500 rpm and the reaction mixture was agitated at ambient temperature. After for 4 h the agitation was stopped and the reaction mixture (organic layer) was analyzed by GC/MS. The crude reaction mixture was shown to contain <5 wt % of the corresponding epoxide. At this time, 0.2 g of the phase-transfer catalyst, (C₄H₉)₄N⁺HSO₄⁻, was added to the reaction mixture and the agitation (1500 rpm) of the reaction mixture was continued at ambient temperature for additional 4 h. Subsequent GC/MS and ¹⁹F-NMR analysis showed that the reaction mixture (organic layer) contained starting material and the corresponding epoxide in a 5:95 ratio, demonstrating 95% conversion of the olefin.

### Example 19

### Comparative Example—Effect of Solvent on Conversion of Perfluoroheptene-3 in Synthesis of 2,3-Difluoro-2-(perfluoroethyl)-3-(perfluoropropyl)oxirane

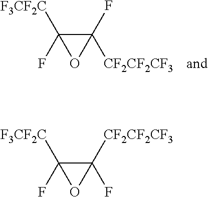

In 20 mL sample vial equipped with magnetic stir bar was added 15 mL of NaOCl solution, 3.5 g of perfluoroheptene-3 (98% purity), and 0.2 g of the phase-transfer catalyst, (C₄H₉)₄N⁺HSO₄⁻. The sample vial was closed, placed on magnetic stir plate, the agitation speed was set to 1500 rpm and the reaction mixture was agitated at ambient temperature. After 4 h the agitation was stopped and the reaction mixture (organic layer) was analyzed by GC/MS. The crude reaction mixture was shown to contain <5 wt % of the corresponding epoxide. At this time, 1 mL of acetonitrile was added to the reaction mixture and the agitation (1500 rpm) was continued at ambient temperature for additional 4 h. Subsequent GC/MS and ¹⁹F-NMR analysis showed that the reaction mixture (organic layer) contained starting material and the corresponding epoxide in a 4:96 ratio, demonstrating 96% conversion of the olefin into the epoxide.

### Example 20

### Comparative Example—Effect of Solvent on Conversion of Olefin in Synthesis of (E)-2,3-bis(perfluorobutyl)oxirane

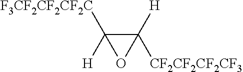

In 20 mL sample vial equipped with magnetic stir bar was placed 15 mL of NaOCl solution, 2.0 g of (E)-1,1,1,2,2,3,3,4,4,7,7,8,8,9,9,10,10,10-octadecafluorodec-5-ene, and 0.2 g of the phase-transfer catalyst, Aliquat® 336. The sample vial was closed, placed on magnetic stir plate, the agitation speed was set to 1500 rpm and the reaction mixture was agitated at ambient temperature. After for 20 h the agitation was stopped and the reaction mixture (organic layer) was analyzed by GC/MS. The crude reaction mixture was shown to contain ˜10 wt % of the corresponding epoxide. At this point 2 mL of acetonitrile was added to the same reaction mixture and the agitation (1500 rpm) of the reaction mixture was continued at ambient temperature for additional 20 h. Subsequent GC/MS and ¹⁹F-NMR analysis showed that the reaction mixture (organic layer) contained starting material and the corresponding epoxide in a 6:94 ratio, demonstrating 94% conversion of the olefin into the epoxide.

### Example 21A

### (E)-2,3-difluoro-2-(trifluoromethyl)oxirane

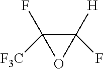

The title compound is prepared by a method analogous to that of Example 1, starting from (E)-1,2,3,3,3-pentafluoropentene (E-HFO-1225ye).

### Example 21B

### (Z)-2,3-difluoro-2-(trifluoromethyl)oxirane

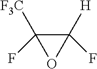

The title compound is prepared by a method analogous to that of Example 1, starting from (Z)-1,2,3,3,3-pentafluoropentene (Z-HFO-1225ye).

### Other Embodiments

1. In some embodiments, the present application provides a process of preparing a compound of Formula (I):

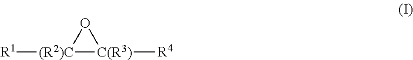

comprising reacting a compound of Formula (II):

R¹—C(R²)═C(R³)—R⁴ (II)

with an aqueous hypohalite salt in the presence of a cationic phase transfer catalyst and an organic solvent, wherein:
R¹ and R⁴ are each independently H, Cl, F, Br, I, a partially fluorinated C₁₋₄ alkoxy, or a perfluorinated C₁₋₄ alkoxy;
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;

wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring.
2. The process of embodiment 1, wherein R¹ and R⁴ are each independently H or F.
3. The process of embodiment 1, wherein R¹ and R⁴ are each independently H or F; and R² and R³ are each independently selected from partially fluorinated or perfluorinated C₁₋₅ alkylene, which together form a monocyclic ring.
4. The process of embodiment 1, wherein R¹ and R⁴ are each independently H or F; and R² and R³ are each independently selected from partially fluorinated or perfluorinated C₁₋₁₀ alkyl.
5. The process of any one of embodiments 1 to 4, wherein the hypohalite salt is selected from NaOCl, KOCl, NaOBr, and Ca(OCl)₂.
6. The process of any one of embodiments 1 to 4, wherein the hypohalite salt is NaOCl.
7. The process of any one of embodiments 1 to 6, wherein the cationic phase transfer catalyst is a quaternary ammonium salt.
8. The process of any one of embodiments 1 to 6, wherein the cationic phase transfer catalyst is a quaternary phosphonium salt.
9. The process of any one of embodiments 1 to 6, the cationic phase transfer catalyst has formula (R^{a})(R^{b})(R^{c})(R^{d})N⁺X⁻ or (R^{a})(R^{b})(R^{c})(R^{d})P⁺X⁻, wherein R^{a}, R^{b}, R^{c}, and R^{d} are each independently selected from C₁₋₁₈ alkyl, C₃₋₁₄ cycloalkyl, 4-14 membered heterocycloalkyl, C₆₋₁₄ aryl, 5-14 membered heteroaryl, C₃₋₁₄ cycloalkyl-C₁₋₃ alkylene, 4-14 membered heterocycloalkyl-C₁₋₃ alkylene, C₆₋₁₄ membered aryl-C₁₋₃ alkylene, and 5-14 membered heteroaryl-C₁₋₃ alkylene, each of which is optionally substituted by 1, 2, 3, or 4 groups independently selected from OH, C₁₋₆ alkoxy, C₁₋₆ alkyl, C₂₋₆ alkenyl, C₁₋₆ fluoroalkyl, di-(C₁₋₆ alkyl)amino, C₃₋₁₄ cycloalkyl, 4-14 membered heterocycloalkyl, C₆₋₁₄ membered aryl, and 5-14 membered heteroaryl; and X⁻ is an anion.
10. The process of any one of embodiments 1 to 6, wherein the cationic phase transfer catalyst has formula (R^{a})(R^{b})(R^{c})(R^{d})N⁺X⁻, wherein R^{a}, R^{b}, R^{c}, and R^{d} are each independently selected from C₁₋₁₂ alkyl; and X is a halide ion or HSO₄⁻.
11. The process of any one of embodiments 1 to 6, wherein the cationic phase transfer catalyst has formula (R^{a})(R^{b})(R^{c})(R^{d})P⁺X⁻, wherein R^{a}, R^{b}, R^{c}, and R^{d} are each independently selected from C₁₋₁₂ alkyl or phenyl; and X is a halide ion or HSO₄⁻.
12. The process of any one of embodiments 1 to 6, wherein the cationic phase transfer catalyst is (n-butyl)₄P⁺Br⁻, (n-butyl)₄P⁺HSO₄⁻, (n-butyl)₄N⁺Br⁻, (n-butyl)₄N⁻HSO₄⁻, Ph₄P⁺Br⁻, (ethyl)₄N⁺Br⁻, or Aliquat® 336.
13. The process of any one of embodiments 1 to 12, wherein the organic solvent is acetonitrile, tetrahydrofuran, diethyl ether, methyl-t-butyl ether, dimethyoxyethane, bis(2-methoxyethyl) ether, or benzene substituted with 1, 2, 3, or 4 independently selected C₁₋₄ alkyl groups.
14. The process of any one of embodiments 1 to 12, wherein the organic solvent is acetonitrile or benzene substituted with 1, 2, 3, or 4 independently selected C₁₋₄ alkyl groups.
15. The process of any one of embodiments 1 to 12, wherein the organic solvent is acetonitrile, toluene, o-xylene, m-xylene, p-xylene or a mixture thereof.
16. The process of any one of embodiments 1 to 4, wherein the hypohalite salt is NaOCl; the cationic phase transfer catalyst is a quaternary phosphonium salt or a quaternary ammonium salt; and the organic solvent is acetonitrile, toluene, o-xylene, m-xylene, p-xylene, or any mixture thereof.
17. The process of any one of embodiments 1 to 16, wherein the reaction is conducted at a pH of from 10 to 14.
18. The process of any one of embodiments 1 to 17, wherein the reaction is conducted at a temperature of from about 10° C. to about 30° C., from about 10° C. to about 20° C., from about 20° C. to about 30° C., or from about 25° C. to about 30° C.
19. The process of any one of embodiments 1 to 18, wherein about 2 to about 4 molar equivalents of the hypohalite salt is used based on one equivalent of the compound of Formula (II).
20. The process of any one of embodiments 1 to 19, wherein about 1 mol % to about 15 mol % of the cationic phase transfer catalyst is used based on one equivalent of the compound of Formula (II).
21. The process of embodiment 1, wherein the compound of Formula (I) is selected from the group consisting of:

(Z)-2,3-difluoro-2-(trifluoromethyl)oxirane;

(E)-2,3-difluoro-2-(trifluoromethyl)oxirane;

cis-2-fluoro-3-(trifluoromethyl)oxirane;

trans-2-fluoro-3-(trifluoromethyl)oxirane;

trans-2,3-bis(trifluoromethyl)oxirane;

cis-2,3-bis(trifluoromethyl)oxirane;

trans-2-(trifluoromethyl)-3-(perfluoroethyl)oxirane;

trans-2-(trifluoromethyl)-3-(perfluoropropan-2-yl)oxirane;

trans-2,3-bis(perfluoropropyl)oxirane;

trans-2-(perfluorobutyl)-3-(perfluoroethyl)oxirane;

trans-2,3-bis(perfluorobutyl)oxirane;

(Z)-2-(2,2,2-Trifluoroethoxy)-3-fluoro-2-(trifluoromethyl)-3-(perfluoroethyl)oxirane;

(E)-2-(2,2,2-Trifluoroethoxy)-3-fluoro-2-(trifluoromethyl)-3-(perfluoroethyl)oxirane;

cis-2,3-dichloro-2,3-bis(trifluoromethyl)oxirane;

trans-2,3-dichloro-2,3-bis(trifluoromethyl)oxirane;

trans-2-fluoro-3-(perfluoropropan-2-yl)oxirane;

cis-2-fluoro-3-(perfluoropropan-2-yl)oxirane;

cis-2,2,3,3,4,4-hexafluoro-6-oxa-bicyclo[3.1.0]hexane;

cis-2,2,3,3-tetrafluoro-5-oxabicyclo[2.1.0]pentane;

cis-2,3-difluoro-2-(perfluoroethyl)-3-(perfluoropropyl)oxirane;

trans-2,3-difluoro-2-(perfluoroethyl)-3-(perfluoropropyl)oxirane;

cis-2,3-difluoro-2-(trifluoromethyl)-3-(perfluoropentyl)oxirane; and

trans-2,3-difluoro-2-(trifluoromethyl)-3-(perfluoropentyl)oxirane.
22. A composition comprising a compound of Formula I:

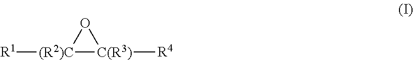

R¹ and R⁴ are each independently H, Cl, F, Br, I, a partially fluorinated C₁₋₄ alkoxy, or a perfluorinated C₁₋₄ alkoxy;

R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;

wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring;

wherein the compound of Formula I has the (Z) or (E) configuration and the composition is substantially free of the opposite stereoisomers.
23. A composition of embodiment 22, wherein the composition comprises a compound selected from:

(Z)-2,3-difluoro-2-(trifluoromethyl)oxirane;

(E)-2,3-difluoro-2-(trifluoromethyl)oxirane;

cis-2-fluoro-3-(trifluoromethyl)oxirane;

trans-2-fluoro-3-(trifluoromethyl)oxirane;

trans-2,3-bis(trifluoromethyl)oxirane;

cis-2,3-bis(trifluoromethyl)oxirane;

trans-2-(trifluoromethyl)-3-(perfluoroethyl)oxirane;

trans-2-(trifluoromethyl)-3-(perfluoropropan-2-yl)oxirane;

trans-2,3-bis(perfluoropropyl)oxirane;

trans-2-(perfluorobutyl)-3-(perfluoroethyl)oxirane;

trans-2,3-bis(perfluorobutyl)oxirane;

(Z)-2-(2,2,2-Trifluoroethoxy)-3-fluoro-2-(trifluoromethyl)-3-(perfluoroethyl)oxirane;

(E)-2-(2,2,2-Trifluoroethoxy)-3-fluoro-2-(trifluoromethyl)-3-(perfluoroethyl)oxirane;

cis-2,3-dichloro-2,3-bis(trifluoromethyl)oxirane;

trans-2,3-dichloro-2,3-bis(trifluoromethyl)oxirane;

trans-2-fluoro-3-(perfluoropropan-2-yl)oxirane;

cis-2-fluoro-3-(perfluoropropan-2-yl)oxirane;

cis-2,2,3,3,4,4-hexafluoro-6-oxa-bicyclo[3.1.0]hexane;

cis-2,2,3,3-tetrafluoro-5-oxabicyclo[2.1.0]pentane;

cis-2,3-difluoro-2-(perfluoroethyl)-3-(perfluoropropyl)oxirane;

trans-2,3-difluoro-2-(perfluoroethyl)-3-(perfluoropropyl)oxirane;

cis-2,3-difluoro-2-(trifluoromethyl)-3-(perfluoropentyl)oxirane; and

trans-2,3-difluoro-2-(trifluoromethyl)-3-(perfluoropentyl)oxirane.
24. A compound selected from the group consisting of:

(Z)-2,3-difluoro-2-(trifluoromethyl)oxirane;

(E)-2,3-difluoro-2-(trifluoromethyl)oxirane;

cis-2-fluoro-3-(trifluoromethyl)oxirane;

trans-2-fluoro-3-(trifluoromethyl)oxirane;

trans-2,3-bis(trifluoromethyl)oxirane;

cis-2,3-bis(trifluoromethyl)oxirane;

trans-2-(trifluoromethyl)-3-(perfluoroethyl)oxirane;

trans-2-(trifluoromethyl)-3-(perfluoropropan-2-yl)oxirane;

trans-2,3-bis(perfluoropropyl)oxirane;

trans-2-(perfluorobutyl)-3-(perfluoroethyl)oxirane;

trans-2,3-bis(perfluorobutyl)oxirane;

(Z)-2-(2,2,2-Trifluoroethoxy)-3-fluoro-2-(trifluoromethyl)-3-(perfluoroethyl)oxirane;

(E)-2-(2,2,2-Trifluoroethoxy)-3-fluoro-2-(trifluoromethyl)-3-(perfluoroethyl)oxirane;

cis-2,3-dichloro-2,3-bis(trifluoromethyl)oxirane;

trans-2,3-dichloro-2,3-bis(trifluoromethyl)oxirane;

trans-2-fluoro-3-(perfluoropropan-2-yl)oxirane;

cis-2-fluoro-3-(perfluoropropan-2-yl)oxirane;

cis-2,2,3,3,4,4-hexafluoro-6-oxa-bicyclo[3.1.0]hexane;

cis-2,2,3,3-tetrafluoro-5-oxabicyclo[2.1.0]pentane;

cis-2,3-difluoro-2-(perfluoroethyl)-3-(perfluoropropyl)oxirane;

trans-2,3-difluoro-2-(perfluoroethyl)-3-(perfluoropropyl)oxirane;

cis-2,3-difluoro-2-(trifluoromethyl)-3-(perfluoropentyl)oxirane; and

trans-2,3-difluoro-2-(trifluoromethyl)-3-(perfluoropentyl)oxirane.
25. A composition comprising a compound of Formula (I):

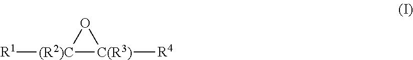

and a compound of Formula (III):

R⁵—C(R⁶)═C(R⁷)—R⁸ (III)

wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are Cl, F, Br, I, partially fluorinated C₁₋₁₀ alkyl, or perfluorinated C₁₋₁₀ alkyl; and
R³ and R⁷ are identical and are partially fluorinated or perfluorinated C₁₋₁₀ alkyl; provided that either R¹ and R⁵ are H or Cl; or R⁴ and R⁸ are H or Cl.
26. A composition comprising a compound of Formula (I):

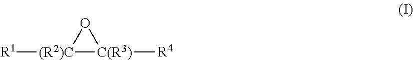

and a compound of Formula (III):

R⁵—C(R⁶)═C(R⁷)—R⁸ (III)

wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are partially fluorinated or perfluorinated C₁₋₅ alkylene; and
R³ and R⁷ are identical and are partially fluorinated or perfluorinated C₁₋₅ alkylene; wherein said R² and R³ are taken together form a monocyclic ring and R⁶ and R⁷ are taken together form a monocyclic ring;
27. The composition of embodiment 25 or 26, wherein the molar ratio of the compound of Formula (I) to the compound of Formula (III) in the composition is from about 50:50 to about 99:0.01
28. The composition of embodiment 25 or 26, wherein the molar ratio of the compound of Formula (I) to the compound of Formula (III) in the composition is from about 80:20 to about 99.9:0.01.
29. The composition of any one of embodiments 25 to 28, wherein the molar ratio of the compound of Formula (I) to the compound of Formula (III) in the composition is from about 90:10 to about 99.99:0.01.
30. The composition of embodiment 25, wherein the composition comprises:
   a compound of Formula (I) which is 2-fluoro-3-(trifluoromethyl)oxirane and a compound of Formula (III) which is 1,3,3,3-tetrafluoroprop-1-ene; or
   a compound of Formula (I) which is 2,3-bis(trifluoromethyl)oxirane and a compound of Formula (III) which is 1,1,1,4,4,4-hexafluorobut-2-ene; or
   a compound of Formula (I) which is 2-(trifluoromethyl)-3-(perfluoroethyl)oxirane and a compound of Formula (III) which is 1,1,1,4,4,5,5,5-octafluoropent-2-ene; or
   a compound of Formula (I) which is 2-(trifluoromethyl)-3-(perfluoropropan-2-yl)oxirane and a compound of Formula (III) which is 1,1,1,5,5,5-hexafluoro-4,4-bis(trifluoromethyl)pent-2-ene; or
   a compound of Formula (I) which is 2,3-bis(perfluoropropyl)oxirane and a compound of Formula (III) which is 1,1,1,2,2,3,3,6,6,7,7,8,8,8-tetradecafluorooct-4-ene; or
   a compound of Formula (I) which is 2-(perfluorobutyl)-3-(perfluoroethyl)oxirane and a compound of Formula (III) which is 1,1,1,2,2,5,5,6,6,7,7,8,8,8-tetradecafluorooct-3-ene; or
   a compound of Formula (I) which is 2,3-bis(perfluorobutyl)oxirane and a compound of Formula (III) which is 1,1,1,2,2,3,3,4,4,7,7,8,8,9,9,10,10,10-octadecafluorodec-5-ene; or
   a compound of Formula (I) which is 2-(2,2,2-Trifluoroethoxy)-3-fluoro-2-(trifluoromethyl)-3-(perfluoroethyl)oxirane and a compound of Formula (III) which is 2-(2,2,2-trifluoroethoxy)-1,1,1,4,4,5,5,5-octafluoropent-2-ene; or
   a compound of Formula (I) which is 2,3-dichloro-2,3-bis(trifluoromethyl)oxirane and a compound of Formula (III) which is 2,3-dichloro-1,1,1,4,4,4-hexafluorobut-2-ene; or
   a compound of Formula (I) which is 2-fluoro-3-(perfluoropropan-2-yl)oxirane and a compound of Formula (III) which is 1,3,4,4,4-pentafluoro-3-(trifluoromethyl)but-1-ene; or
   a compound of Formula (I) which is 2,3-difluoro-2-(perfluoroethyl)-3-(perfluoropropyl)oxirane and a compound of Formula (III) which is perfluoroheptene-3; or
   a compound of Formula (I) which is 2,3-difluoro-2-(trifluoromethyl)-3-(perfluoropentyl)oxirane and a compound of Formula (III) which is perfluoroctene-2.
31. The composition of embodiment 26, wherein the composition comprises:
   a compound of Formula (I) which is 2,2,3,3,4,4-hexafluoro-6-oxa-bicyclo[3.1.0]hexane and a compound of Formula (III) which is 3,3,4,4,5,5-hexafluorocyclopent-1-ene; or
   a compound of Formula (I) which is 2,2,3,3-tetrafluoro-5-oxabicyclo[2.1.0]pentane and a compound of Formula (III) which is 3,3,4,4-tetrafluorocyclobut-1-ene.
32. The composition of any one of embodiments 25 to 26, wherein the compounds of Formula (I) and (III) are cis-isomers.
33. The composition of any one of embodiments 25, wherein the compounds of Formula (I) and (III) are trans-isomers.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A process of preparing a compound of Formula (I): 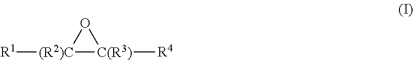
comprising reacting a compound of Formula (II):
R¹—C(R²)═C(R³)—R⁴ (II)
with an aqueous hypohalite salt in the presence of a cationic phase transfer catalyst and an organic solvent, wherein:
R¹ and R⁴ are each independently H, Cl, F, Br, I, a partially fluorinated C₁₋₄ alkoxy, or a perfluorinated C₁₋₄ alkoxy;
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring.

2. (canceled)

3. (canceled)

4. The process of claim 1, wherein R¹ and R⁴ are each independently H or F; and R² and R³ are each independently selected from partially fluorinated or perfluorinated C₁₋₁₀ alkyl.

5. The process of claim 1, wherein the hypohalite salt is selected from NaOCl, KOCl, NaOBr, and Ca(OCl)₂.

6. (canceled)

7. The process of claim 1, wherein the cationic phase transfer catalyst is a quaternary ammonium salt or a quaternary phosphonium salt.

8. (canceled)

9. The process of claim 1, the cationic phase transfer catalyst has formula (R^{a})(R^{b})(R^{c})(R^{d})N⁺X⁻ or (R^{a})(R^{b})(R^{c})(R^{d})P⁺X⁻, wherein R^{a}, R^{b}, R^{c}, and R⁴ are each independently selected from C₁₋₁₈ alkyl, C₃₋₁₄ cycloalkyl, 4-14 membered heterocycloalkyl, C₆₋₁₄ aryl, 5-14 membered heteroaryl, C₃₋₁₄ cycloalkyl-C₁₋₃ alkylene, 4-14 membered heterocycloalkyl-C₁₋₃ alkylene, C₆₋₁₄ membered aryl-C₁₋₃ alkylene, and 5-14 membered heteroaryl-C₁₋₃ alkylene, each of which is optionally substituted by 1, 2, 3, or 4 groups independently selected from OH, C₁₋₆ alkoxy, C₁₋₆ alkyl, C₂₋₆ alkenyl, C₁₋₆ fluoroalkyl, di-(C₁₋₆ alkyl)amino, C₃₋₁₄ cycloalkyl, 4-14 membered heterocycloalkyl, C₆₋₁₄ membered aryl, and 5-14 membered heteroaryl; and X⁻ is an anion.

10. The process of claim 1, wherein the cationic phase transfer catalyst has formula (R^{a})(R^{b})(R^{c})(R^{d})N⁺X⁻, wherein R^{a}, R^{b}, R^{c}, and R⁴ are eah independently selected from C₁₋₁₂ alkyl; and X is a halide ion or HSO₄⁻.

11. The process of claim 1, wherein the cationic phase transfer catalyst has formula (R^{a})(R^{b})(R^{c})(R^{d})P⁺X⁻, wherein R^{a}, R^{b}, R^{c}, and R^{d} are each independently selected from C₁₋₁₂ alkyl or phenyl; and X is a halide ion or HSO₄⁻.

12. (canceled)

13. The process of claim 1, wherein the organic solvent is acetonitrile, tetrahydrofuran, diethyl ether, methyl-t-butyl ether, dimethyoxyethane, bis(2-methoxyethyl) ether, or benzene substituted with 1, 2, 3, or 4 independently selected C₁₋₄ alkyl groups.

14. The process of claim 1, wherein the organic solvent is acetonitrile or benzene substituted with 1, 2, 3, or 4 independently selected C₁₋₄ alkyl groups.

15. The process of claim 1, wherein the organic solvent is acetonitrile, toluene, o-xylene, m-xylene, p-xylene or a mixture thereof.

16. (canceled)

17. The process of claim 1, wherein the reaction is conducted at a pH of from 10 to 14.

18. The process of claim 1, wherein the reaction is conducted at a temperature of from about 10° C. to about 30° C., from about 10° C. to about 20° C., from about 20° C. to about 30° C., or from about 25° C. to about 30° C.

19. (canceled)

20. (canceled)

21. The process of claim 1, wherein the compound of Formula (I) is selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

22. A composition comprising a compound of Formula I: 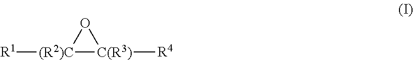
R¹ and R⁴ are each independently H, Cl, F, Br, I, a partially fluorinated C₁₋₄ alkoxy, or a perfluorinated C₁₋₄ alkoxy;
R² is selected from H, Cl, F, Br, I, a partially fluorinated C₁₋₁₀ alkyl, a perfluorinated C₁₋₁₀ alkyl, a partially fluorinated C₁₋₄ alkoxy, and a perfluorinated C₁₋₄ alkoxy;
wherein at least one of R¹, R², and R⁴ is not H; and
R³ is selected from partially fluorinated and perfluorinated C₁₋₁₀ alkyl;
or alternatively, R² and R³ are each independently selected from partially fluorinated or perfluorinated C₁₋₅ alkylene, which together form a monocyclic ring;
wherein the compound of Formula I has the (Z) or (E) configuration and the composition is substantially free of the opposite stereoisomers.

23. A composition of claim 22, wherein the composition comprises a compound selected from:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

24. A compound selected from the group consisting of:
(Z)-2,3-difluoro-2-(trifluoromethyl)oxirane;
(E)-2,3-difluoro-2-(trifluoromethyl)oxirane;
cis-2-fluoro-3-(trifluoromethyl)oxirane;
trans-2-fluoro-3-(trifluoromethyl)oxirane;
trans-2,3-bis(trifluoromethyl)oxirane;
cis-2,3-bis(trifluoromethyl)oxirane;
trans-2-(trifluoromethyl)-3-(perfluoroethyl)oxirane;
trans-2-(trifluoromethyl)-3-(perfluoropropan-2-yl)oxirane;
trans-2,3-bis(perfluoropropyl)oxirane;
trans-2-(perfluorobutyl)-3-(perfluoroethyl)oxirane;
trans-2,3-bis(perfluorobutyl)oxirane;
(Z)-2-(2,2,2-Trifluoroethoxy)-3-fluoro-2-(trifluoromethyl)-3-(perfluoroethyl)oxirane;
(E)-2-(2,2,2-Trifluoroethoxy)-3-fluoro-2-(trifluoromethyl)-3-(perfluoroethyl)oxirane;
cis-2,3-dichloro-2,3-bis(trifluoromethyl)oxirane;
trans-2,3-dichloro-2,3-bis(trifluoromethyl)oxirane;
trans-2-fluoro-3-(perfluoropropan-2-yl)oxirane;
cis-2-fluoro-3-(perfluoropropan-2-yl)oxirane;
cis-2,2,3,3,4,4-hexafluoro-6-oxa-bicyclo[3.1.0]hexane;
cis-2,2,3,3-tetrafluoro-5-oxabicyclo[2.1.0]pentane;
cis-2,3-difluoro-2-(perfluoroethyl)-3-(perfluoropropyl)oxirane;
trans-2,3-difluoro-2-(perfluoroethyl)-3-(perfluoropropyl)oxirane;
cis-2,3-difluoro-2-(trifluoromethyl)-3-(perfluoropentyl)oxirane; and
trans-2,3-difluoro-2-(trifluoromethyl)-3-(perfluoropentyl)oxirane.

25. A composition comprising a compound of Formula (I): 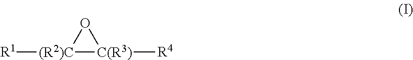
and a compound of Formula (III):
R⁵—C(R⁶)═C(R⁷)—R⁸ (III)
wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are Cl, F, Br, I, partially fluorinated C₁₋₁₀ alkyl, or perfluorinated C₁₋₁₀ alkyl; and
R³ and R⁷ are identical and are partially fluorinated or perfluorinated C₁₋₁₀ alkyl; provided that either R¹ and R⁵ are H or Cl; or R⁴ and R⁸ are H or Cl.

26. A composition comprising a compound of Formula (I): 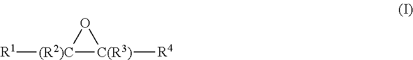
and a compound of Formula (III):
R⁵—C(R⁶)═C(R⁷)—R⁸ (III)
wherein:
the compounds of Formula (I) and Formula (III) have the same stereochemistry;
R¹ and R⁵ are identical and are H, Cl, F, Br, I, a partially fluorinated C₁₋₄ alkoxy, or a perfluorinated C₁₋₄ alkoxy;
R⁴ and R⁸ are identical and each independently H, Cl, F, Br, I, a partially fluorinated C₁₋₄ alkoxy, or a perfluorinated C₁₋₄ alkoxy;
R² and R⁶ are identical and are partially fluorinated or perfluorinated C₁₋₅ alkylene; and
R³ and R⁷ are identical and are partially fluorinated or perfluorinated C₁₋₅ alkylene; wherein said R² and R³ are taken together form a monocyclic ring and R⁶ and R⁷ are taken together form a monocyclic ring;

27. The composition of claim 25, wherein the molar ratio of the compound of Formula (I) to the compound of Formula (III) in the composition is from about 50:50 to about 99.:0.01

28. (canceled)

29. (canceled)

30. The composition of claim 25, wherein the composition comprises:
a compound of Formula (I) which is 2-fluoro-3-(trifluoromethyl)oxirane and a compound of Formula (III) which is 1,3,3,3-tetrafluoroprop-1-ene; or
a compound of Formula (I) which is 2,3-bis(trifluoromethyl)oxirane and a compound of Formula (III) which is 1,1,1,4,4,4-hexafluorobut-2-ene; or
a compound of Formula (I) which is 2-(trifluoromethyl)-3-(perfluoroethyl)oxirane and a compound of Formula (III) which is 1,1,1,4,4,5,5,5-octafluoropent-2-ene; or
a compound of Formula (I) which is 2-(trifluoromethyl)-3-(perfluoropropan-2-yl)oxirane and a compound of Formula (III) which is 1,1,1,5,5,5-hexafluoro-4,4-bis(trifluoromethyl)pent-2-ene; or
a compound of Formula (I) which is 2,3-bis(perfluoropropyl)oxirane and a compound of Formula (III) which is 1,1,1,2,2,3,3,6,6,7,7,8,8,8-tetradecafluorooct-4-ene; or
a compound of Formula (I) which is 2-(perfluorobutyl)-3-(perfluoroethyl)oxirane and a compound of Formula (III) which is 1,1,1,2,2,5,5,6,6,7,7,8,8,8-tetradecafluorooct-3-ene; or
a compound of Formula (I) which is 2,3-bis(perfluorobutyl)oxirane and a compound of Formula (III) which is 1,1,1,2,2,3,3,4,4,7,7,8,8,9,9,10,10,10-octadecafluorodec-5-ene; or
a compound of Formula (I) which is 2-(2,2,2-Trifluoroethoxy)-3-fluoro-2-(trifluoromethyl)-3-(perfluoroethyl)oxirane and a compound of Formula (III) which is 2-(2,2,2-trifluoroethoxy)-1,1,1,4,4,5,5,5-octafluoropent-2-ene; or
a compound of Formula (I) which is 2,3-dichloro-2,3-bis(trifluoromethyl)oxirane and a compound of Formula (III) which is 2,3-dichloro-1,1,1,4,4,4-hexafluorobut-2-ene; or
a compound of Formula (I) which is 2-fluoro-3-(perfluoropropan-2-yl)oxirane and a compound of Formula (III) which is 1,3,4,4,4-pentafluoro-3-(trifluoromethyl)but-1-ene; or
a compound of Formula (I) which is 2,3-difluoro-2-(perfluoroethyl)-3-(perfluoropropyl)oxirane and a compound of Formula (III) which is perfluoroheptene-3; or
a compound of Formula (I) which is 2,3-difluoro-2-(trifluoromethyl)-3-(perfluoropentyl)oxirane and a compound of Formula (III) which is perfluoroctene-2.

31. The composition of claim 26, wherein the composition comprises:
a compound of Formula (I) which is 2,2,3,3,4,4-hexafluoro-6-oxa-bicyclo[3.1.0]hexane and a compound of Formula (III) which is 3,3,4,4,5,5-hexafluorocyclopent-1-ene; or
a compound of Formula (I) which is 2,2,3,3-tetrafluoro-5-oxabicyclo[2.1.0]pentane and a compound of Formula (III) which is 3,3,4,4-tetrafluorocyclobut-1-ene.

32. (canceled)

33. (canceled)
